(19) United States
(12) Patent Application Publication
(10) Pub. No.: US 2004/0198681 A1
(43) Pub. Date: Oct. 7, 2004

(54) MB-1 analogs and uses thereof

(75) Inventors: Marc Beauregard, Cap-Sante (CA); Mylene-Claude Gagnon, Cap-Sante (CA); Alain Doucet, L'Ancienne-Lorette (CA); Martin Williams, Charny (CA)

Correspondence Address: OGILVY RENAULT, 1981 MCGILL COLLEGE AVENUE, SUITE 1600, MONTREAL, QC, H3A2Y3, CA

(73) Assignee: AGROTERRA BIOTECH INC., Trois-Rivieres (CA)

(21) Appl. No.: 10/625,882

(22) Filed: Jul. 24, 2003

Related U.S. Application Data
(63) Continuation-in-part of application No. 10/272,929, filed on Oct. 18, 2002.
(60) Provisional application No. 60/329,759, filed on Oct. 18, 2001.

(51) Int. Cl. A61K 31/70; A01N 43/04; C12N 15/00; C12N 15/09; C12N 15/63; C12N 15/70; C12N 15/74; C07K 1/00; C07K 14/00; C07K 17/00; A01N 37/18; A61K 38/00; C12N 5/00; C12N 5/02

(52) U.S. Cl. 514/44; 435/320.1; 530/350; 514/2; 435/325

(57) ABSTRACT

The present invention relates to protein engineering for production of analogs of MB-1. MB-1 analogs are more stable to different biochemical conditions than the native protein, and allow for improving the supplying of essential amino acids to human and animals. De novo synthesis of MB-1 analogs is principally targeted to enhance availability of essential amino acids required for farm animal production or human health.

### CROSS-REFERENCES TO RELATED APPLICATION

The present application is a Continuation-in-part of U.S. patent application Ser. No. 10/272,929 filed on Oct. 18, 2002, which claims priority of U.S. provisional patent application serial No. 60/329,759 filed Oct. 10, 2001.

### BACKGROUND OF THE INVENTION

1. Field of the Invention

The invention relates to protein design or protein engineering in the production of polypeptides allowing delivery of food essential amino acids. This invention particularly relates to de novo synthesis of polypeptides designed to enhance availability of amino acids required for milk production in cows, farm animal production or human food.

2. Description of Prior Art

Diet is important in determining general health, work performance, energy level and appearance. An equilibrated diet should include proper amounts of essential nutrients, which are the nutritive elements that an animal is incapable of synthesizing by itself and therefore, that must be obtained from food. As regimen is highly variable throughout animal kingdom, essential nutrient requirements are significantly different for each animal specie. In humans, approximately forty essential nutrients are required, wherein fourteen are minerals, fourteen are vitamins, ten are amino acids and two are essential fatty acids.

Other non-essential, but beneficial, elements can also be obtained from food, including enzymes, beneficial bowel bacteria and certain kinds of fiber. Ultimately, supplying these factors can become essential, especially when the diet contains highly processed foods. Indeed, extensively processed foods fail to provide all the nutritional elements required for optimum animal or human health since processing destroys the chemically fragile essential nutritional elements. For example, processing steps may expose the carbon-carbon double bonds in the hydrocarbon chains of essential fatty acids to oxidizing agents such as light and oxygen, which cause the lost of their nutritional value.

Despite their publicized deficiencies, processed aliments have become popular due to the ease, speed and convenience with which meals can be prepared. As a result of a diet rich in such aliments, humans and animals often suffer from a nutritional deficiencies. This reality poses a particular problem in animal feeding strategies since many of the commonly available blends of feed tend to be somewhat deficient in certain key amino acids. As a result, the production of amino acid supplements for enhancing animal feed use has become an important worldwide business.

The amino acid supplements can be produced either by fermentation or by chemical means. These pure (highly variable) amino acids or amino acid analogs are then added at the required levels to provide a balanced protein diet for feeding the animal. As example, the amino acid methionine, the aqueous solutions of methionine salts, in particular sodium methionine, and substitutes such as the methionine hydroxy analogue (MHA) are used all over the world as feed additives for breeding poultry, pigs and other economically useful animals, and to stimulate the production of animal protein. In addition to its use in meat production purpose, methionine play a role in milk production, being a limiting amino acid for milk protein production. A well balanced level of methionine is therefore thought to result in effective levels of milk protein production and in an increase in milk production.

To benefit from these advantages, methionine can be added directly to animals food. However, the native form of methionine is rapidly degraded by bacteria in the rumen of bovine and, consequently, only a small portion of the methionine enters the bloodstream. To overcome this problem, a general strategy consisting in introducing methionine into the diet in a protected or modified form, allows the compound to pass through the rumen and to remain unaffected. The methionine is then released from the protected or modified form and passes trough the small intestine, being further absorbed into the bloodstream.

One of the most widely studied compounds for this particular purpose is the hydroxy analogue of methionine 2-hydroxy-4-(methylthio) butanoic acid. International Patent application published under number WO 99/04647 discloses a method for introducing methionine into the rumen by supplementing the feed with an hydroxy analogue of methionine. In this patent application, it is claimed that the hydroxy analogue is substantially unaffected by rumen degradation, passing through the rumen and consequently providing at least 20%, preferably at least 40% of the hydroxy analogue for absorption into the bloodstream through the intestine. The patent application refers to the hydroxy analogue, its salts, esters, amides and oligomers as being “rumen by-pass” and claims an improved efficient means of introducing methionine into the bloodstream of the cow. It is disclosed that the compounds by-pass the rumen and are absorbed in the intestine.

In spite of the large manufacturing capacity for AA it remains that they represent a large portion of costs associated to feeding animal The expense associated with feed additives used for animal production led to the study of intracellular production of high-quality protein by transgenic crops and other organisms, as mean for obtaining efficient and low cost sources of essential amino acids (EAA).

One reason why we should develop other sources of methionine is that current sources of Met are contaminated by equivalent amounts of D-Met, while only L-Met contributes to enhancement of performances. Note that other AA are obtained from fermentation, and are free of D-isomer. Another advantage is that by using plants to produce proteins enriched in AA, one provides animal with a more natural source of AA (i.e. a complete protein) and can use the protein to provide other important AA, represented in tailored ratios. The importance of adjusting ratios of EAA such as Lys, Thr, Trp in addition to Met is now widely recognized. Such mixtures are more efficient than Met alone.

Three main approaches for improving protein quality in a given organism have been explored. The first approach involves transferring a gene coding for a high-quality protein from one organism to another that is more suitable for farming practices (heterologous expression). Recently, soybean and sunflower albumins have been chosen for their high methionine contents for the development of transgenic crops by major agro-biotechnology companies. With this approach, the amino acid composition of a natural protein is naturally predetermined and may not be conformed to the desired EAA ratio. The second approach involves modifying the genes of an organism so that specified EAAs are incorporated into the proteins. This strategy, however, often destabilize the protein and/or prevent it from folding, which may jeopardize its recovery.

U.S. Pat. No. 6,169,232 describes tryptophan enriched plants that have been genetically engineered. Despite the result achieved, it remains that previous protein engineering projects focused on a single EAA at a time, in the context of a natural protein with a given composition.

The last approach involves creating a new protein with a biased composition of selected EAAs, an approach commonly called de novo design. Theoretically, this strategy allows for full control of the amino acid composition of the protein and is, thus, an advantage over the previously mentioned options. De novo design of artificial proteins is an emerging area of research that rely on the understanding of protein structure to allow the creation of molecules with desirable and specific structures and properties. De novo protein design received considerable attention in the last years, allowing significant advances to be made in the attempt to reach the goal of producing stable, well-folded proteins with novel sequences.

Efforts to design proteins are based on knowledge of the physical properties that determine protein structure, such as the patterns of hydrophobic and hydrophilic residues in the sequence, salt bridges bonds, hydrogen bonds, and secondary structural preferences of amino acids.

Various approaches to apply these principles have been attempted. For example, the construction of alpha-helical and beta-sheet proteins with native-like sequences was attempted by individually selecting the residue required at every position in the target fold. Alternatively, a minimalist approach was used to design helical proteins, where the simplest possible sequence believed to be consistent with the folded structure was generated, with varying degrees of success. An experimental method that relies on the hydrophobic and polar (HP) pattern of a sequence was developed where a library of sequences with the correct pattern for a four helix bundle was generated by random mutagenesis. Among non de novo approaches, domains of naturally occurring proteins have been modified or coupled together to achieve a desired tertiary or quaternary organization.

Though the correct secondary structure and overall tertiary organization seem to have been attained by several of the above techniques, many designed proteins appear to lack the structural specificity of native proteins. The complementary geometric arrangement of amino acids in the folded protein is the root of this specificity and is encoded in the sequence.

Recent studies using coiled coils have demonstrated that core side-chain packing can be combined with explicit backbone flexibility. In these cases, the goal was to search for backbone coordinates that satisfied a fixed amino acid sequence. Recently, an algorithm for precise engineering of protein cores was reported.

It would be highly desirable to be provided with new engineered polypeptides capable of acting as amino acid delivery system. It would be desirable also to be provided with new engineered protein that would be a source of selected EAA in given proportions, and be sufficiently stable as to resist degradation in a production host (bacteria or plant).

### SUMMARY OF THE INVENTION

One object of the present invention is to provide a method for improving the supplying of at least one essential amino acid to a human or an animal, which comprises administering to a human or an animal at least one polypeptide having an amino acid sequence selected from the group consisting of SEQ ID NO:1, SEQ ID NO:2, SEQ ID NO:3, SEQ ID NO:4, and SEQ ID NO:5, or a fragment thereof between 10 to 90 amino acids, or an encoding nucleic acid thereof, which may be a DNA or a RNA.

The polypeptide can be administered orally to a human or an animal.

The essential amino acid may be a methionine, lysine, threonine, leucine, tryptophan, an analog or a derivative thereof. The animal may be selected from a mammal, a bird, or a fish.

Another object of the present invention is to provide a composition comprising at least one polypeptide having an amino acid sequence selected from the group consisting of SEQ ID NO:1, SEQ ID NO:2, SEQ ID NO:3, SEQ ID NO:4, and SEQ ID NO:5 for endowing a human or an animal with at least one essential amino acid by oral administration.

One object of the invention is to provide a nucleotide sequence selected from the group consisting of SEQ ID NO:6, SEQ ID NO:7, SEQ ID NO:8, SEQ ID NO:9 and SEQ ID NO:10, or any other nucleic acid sequence under form of a DNA or a RNA, coding for at least one polypeptide consisting of an amino acid sequence selected from the group consisting of SEQ ID NO:1, SEQ ID NO:2, SEQ ID NO:3, SEQ ID NO:4 and SEQ ID NO:5, or an analog or fragment thereof.

Also, in accordance with the present invention there is provided an expression vector comprising a nucleotide sequence selected from the group consisting of SEQ ID NO:6, SEQ ID NO:7, SEQ ID NO:8, SEQ ID NO:9 and SEQ ID NO:10, designed for the production of a recombinant polypeptide consisting of the amino acid sequence as set forth in SEQ ID NO:1, SEQ ID NO:2, SEQ ID NO:3, SEQ ID NO:4, and SEQ ID NO:5, the recombinant peptide being produced in a bacteria, yeast or an eukaryotic cell.

Another object of the invention is to provide a cell transformed with the expression vector comprising a nucleotide sequence selected form the group consisting of SEQ ID NO:6, SEQ ID NO:7, SEQ ID NO:8, SEQ ID NO:9 and SEQ ID NO:10.

Also, another object of the present invention is to provide a method for improving the supplying of at least one essential amino acid to a human or an animal, or for endowing a human or an animal with at least one essential amino acid, comprising administering to the human or animal at least one polypeptide or a nucleic acid sequence encoding the polypeptide, the polypeptide having the amino acid sequence consisting of at least one of SEQ ID NO:1 to SEQ ID NO:5 or a fragment thereof, in which at least one leucine at position 19, 45 or 68, one asparagine at position 44, or one alanine at position 31, 35, 59, 80, or 84 of the SEQ ID NO:1 to SEQ ID NO:5 is substituted with a different amino acid. The functional fragment may comprise between 10 and 90 amino acids.

Preferentially, a leucine can be substituted with a glutamic acid, an aspartic acid, a valine, an isoleucine, a methionine, or a phenylalanine, the asparagine can substituted with a glutamic acid, or an alanine can be substituted with a valine, an isoleucine, a phenylalanine, a leucine, or a methionine.

In accordance with the present invention, there is also provided composition for improving supplying of at least one essential amino acid to a human or an animal or endowing a human or an animal with at least one essential amino acid, the composition comprising at least one polypeptide having amino acid sequence consisting of at least one of SEQ ID NO:1 to SEQ ID NO:5 or a fragment thereof, or a nucleic acid sequence encoding the polypeptide or fragment thereof, having at least one substituted amino acid as described herein.

Preferentially, the essential amino acid is a methionine, lysine, threonine, leucine, tryptophan, arginine, an analog or a derivative thereof.

One object of the present invention is also to provide a polypeptide consisting of an amino acid sequence selected from the group consisting of SEQ ID NO:1 to SEQ ID NO:5 or a functional fragment thereof, in which at least one leucine at position 19, 45 or 68, one asparagine at position 44, or one alanine at position 31, 35, 59, 80, or 84 of at least one of the SEQ ID NO:1 to SEQ ID NO:5 is substituted with a different amino acid.

Still, there is provided a nucleotide sequence selected from the group consisting of, SEQ ID NO:6 to SEQ ID NO:10, in which at least one codon is mutated to allow synthesis from said nucleotide sequence a polypeptide as described herein.

According to another object of the invention, there is provided a method for improving a physical, a biochemical or a biological property of a polypeptide having amino acid sequence consisting of at least one of SEQ ID NO:1 to SEQ ID NO:5 or a fragment thereof, comprising substituting at least one leucine at position 19, 45 or 68, one asparagine at position 44, or one alanine at position 31, 35, 59, 80, or 84 of at least one of SEQ ID NO:1 to SEQ ID NO:5 with a different amino acid. The property can be resistance to temperature as well as resistance to proteolysis.

### DESCRIPTION OF THE DRAWINGS

FIG. 1 illustrates MB-1's hydrophobic core as predicted by design;

FIG. 2 illustrates a circular dichroism spectra of MB-1Trp;

FIG. 3 illustrates the unfolding of MB-1Trp as monitored by fluorospectroscopy;

FIG. 4 illustrates the enhancement of ANSA fluorescence by MB-1Trp;

FIG. 5 illustrates a thermal denaturation curve for MB-1Trp;

FIG. 6 illustrates the degradation of MB-1 and MB-1 Trp mutant using Pronase E;

FIG. 7 illustrates a schematic representation of MB-1LH and MB-1RH;

FIG. 8 illustrates CD spectra of the four mutants;

FIG. 9 illustrates CD spectra measured in the presence of DTT;

FIG. 10 illustrates examples of thermal denaturation curves;

FIG. 11 illustrates the proteolytic degradation of the proteins;

FIG. 12 illustrates an SDS-PAGE analysis of MB-1TrpRH;

FIG. 13 illustrates a circular dichroism spectra of MB-1 mutants;

FIG. 14 illustrates thermal denaturation curves for MB-1RH and MB-1Trp.

FIG. 15 illustrates a design of MB-1RH;

FIG. 16 illustrates the degradation of MB-1(WT) and mutants by pronase E;

FIG. 17 illustrates the predicted location of secondary structures of MB-1Trp;

FIG. 18 illustrates the circular dichroism spectra of MB-1Trp and its mutants;

FIG. 19 illustrates tryptophan fluorescence spectra of MB-1Trp and its variants;

FIG. 20 illustrates the thermal denaturation of MB-1Trp and its variants monitored using molar ellipticity at 222 nm;

FIG. 21 illustrates the proteolytic degradation of MB-1Trp and its variants based on the protein disappearance during incubation with proteases;

FIG. 22 illustrates the predicted position of buried residues in the background, and the exposed residues of the four helices in one plane at the forefront, highlighting the positions of sensitive bonds in helices 2 and 4; and

FIG. 23 illustrates the western blot analysis of the proteolytic resistance of the protein and mutants performed after 0 and 30 minutes exposure to E.coli proteases.

### DETAILED DESCRIPTION OF THE PREFERRED EMBODIMENT

In accordance with the present invention, there is provided a method for supplying at least one essential amino acid (EAA) to a human or an animal, by administering to the human or the animal at least one polypeptide having an amino acid sequence selected from the group consisting of SEQ ID NO:1, SEQ ID NO:2, SEQ ID NO:3, SEQ ID NO:4, and SEQ ID NO:5. The polypeptides of the invention may be delivered to a human or an organisms by oral, intravenous, intraperitoneal, or percutaneous administration. Although a full length polypeptide is preferred, functional fragments are also included in the present invention. Functional fragment comprises between 10 and 90 amino acids from polypeptides of SEQ ID NO:1, SEQ ID NO:2, SEQ ID NO:3, SEQ ID NO:4 or SEQ ID NO:5, where they can be product from full length polypeptide processing. Also, the functional fragment can be produced from expression of truncated nucleic acid sequences SEQ ID NO:6, SEQ ID NO:7, SEQ ID NO:8, SEQ ID NO:9 and SEQ ID NO:10.

The polypeptides described herein have the particularity of being composed of high ratios of amino acids that are essential to human and animal nutrition. A food for specified health use (the so-called physiologically functional food) endowed with a function of delivering EAA through addition of EAA giving polypeptides in an organism, can be prepared by using the polypeptide of the invention or the fraction containing the polypeptide as an active component. And the polypeptide of the invention can be used as a food additive of general foods.

The kinds of the above food are not particularly limited. The physiologically functional food may be applicable to any human food, as for example, but not limited to, milk, pudding, curry, stew, meat sauce, ham, cake, chocolate and the like. In particular, milk is preferable since it can facilitate the intake of the polypeptide of the invention which is difficult for infants. Also, the physiologically functional food formed by addition of the polypeptides of the invention may be given to animals under solid or liquid form. Farm animals would particularly benefit of such a food. The amount of the polypeptide of the invention added to the physiologically functional food is appropriately selected depending on the kind of the food, the purpose of addition of the polypeptide of the invention, the effect expected to be produced by the intake of the food, etc.

In one embodiment of the present invention, the polypeptide can be consumed as a nutraceutical compound, i.e. the protein does not require to be consumed as an aliment. The polypeptide can be produced from a cell or an organisms that have dietary properties and further extracted or concentrated to be presented as a pill, a powder, a potion or any other pharmaceutical forms that are commonly no associated to an aliment. The processed EAA-containing polypeptide can therefore be administered to a human or an animal as a food supplement, indistinctively of food consumption.

In one embodiment of the present invention, the animal species may be selected from mammal, birds or fish. In humans, the essential amino acid can be histidine, leucine, threonine, lysine, tryptophan, phenylalanine, valine, methionie or isoleucine, the 9 amino acids that are not synthesized by human cells. However, in humans, as well as in other animal species, methionine, lysine, threonine, leucine, tryptophan, arginine, and analogs or derivatives thereof are the preferred amino acids to be supplied. As a mammal, the human could beneficiate from an amino acid enriched diet in countries where food availability and quality is reduced. As well, in Western countries, excessive intake of fat and sugar is known to cause obesity, hyperlipidemia and the like. The occurrence of different health disorders is linked to feeding with bad food products or by insufficiently equilibrated food. For example, elevations of triglyceride (TG) levels in blood in hyperlipidemia are said to become a cause which brings disorders such as hypertension and arteriosclerosis. Then, a number of attempts to inhibit elevations of TG levels in blood have been made to improve obesity and hyperlipidemia. The issues of being able to keep foods provided to human equilibrated depends most of the time on the bioavailability of the different components thereof. Therefore, food supplementation with high EAA-content proteins would help to keep equilibrated the diet of these populations.

The polypeptides described herein could receive attention for feeding purpose of poultry species as well as other breaded species grown for human consumption purposes. The feed in which the polypeptide of the invention is combined may be either a feed for livestock such as cows, pigs, chickens, etc. or a feed for hatchery fish such as sea breams, young yellowtails, etc.; the kind of the feed is not particularly limited. The amount of the polypeptide of the invention combined in a feed is appropriately selected depending on the kind of the feed, the effect expected to be produced by the intake of the feed, etc. Generally, it is preferable that the polypeptide of the invention be combined in a feed at the ratio of 0.01 to 0.5% by weight.

A feed endowed with a function of delivery of EAA in livestock, etc. can be prepared by combining in a feed the polypeptide of the invention or the fraction containing the polypeptide as an active component.

In the present invention, the limiting amino acids required for milk protein production were particularly targeted, since one application of the invention is food supply for milk cow breeding. Typical dairy cow diets are limiting in the amino acids lysine and methionine. Methionine is important in the synthesis of milk fat, whereas lysine would play an important role in the amino acid metabolism in mammary gland of cow. Previous research showed that milk production is increased by the addition of rumen protected lysine and methionine when feeding the animal with feed proteins. However, adding feed protein also increases the availability of other amino acids and therefore more nitrogen to the animal than needed.

In another embodiment of the present invention, there is provided a new synthetic protein with a biased composition of selected essential amino acids. Synthetic amino acid sequence synthesis allows for a full control of the amino acid composition of the protein and is, thus, an advantage over generating mutations into naturally occurring proteins. To achieve supplementation of food with amino acids, a strategy consisting in the production of high EAA content synthetic polypeptide, such as milk bundle 1 protein (MB-1), has been explored. Characterisation of MB-1 indicated that the design process used results in the stable expression of a new, largely helical protein enriched in the selected essential amino acids (60% in M, T, K and L). After a first round of design, the protein MB-1 was found to have a folded core and low affinity for 8-anilino-1-naphthalenesulfonic acid (ANSA). Its behaviour and expression levels in vivo were found to be far superior to earlier attempts in the area of high essential amino acid polypeptide design. However, investigation of MB-1's properties did also reveal some flaws. The proteins appear to associate into dimers that could dissociate into monomers in the presence of a high salt concentration. As well, its melting temperature was found to be very low (39° C.), and its resistance to proteases at a physiological temperature was also found to be limited, a possible consequence of partial unfolding. As a consequence of these weaknesses, efforts in growing crystals were unsuccessful.

One way for improving the properties of MB-1 is to increase its conformational stability, which often correlates with resistance to proteolytic degradation. To improve MB-1's stability, additional design cycle were studied. In the absence of an X-ray-resolved structure, extensive core redesign and elaborated fold-specifying devices had to be ruled out. In order to improve the properties of new proteins or peptides of the present invention, dominant folding principles were identified in selected natural proteins and encoded into the new amino acid sequence. By conferring structure, compactness and stability, a protein normally stable in vivo is designed, regardless of its biased composition. In view of its apparent simplicity, we chose the insertion of disulfide bridges. This strategy was chosen not only for its simplicity but for other reasons as well: (1) an intramolecular disulfide bridge would bring stability to the protein by reducing the entropy of the unfolded state; (2) the disulfide bridge is a well-known stabilizer against proteolysis, probably due to its impact on target access by proteases; (3) its engineering involves only a small modification of the amino acid composition, which is critical in order for MB-1 to remain nutritionally efficient.

According to another embodiment of the present invention, analogs of milk bundle-1 (MB-1) protein have been developed. These analogs were developed to overcome the flaws mentioned above that were observed with MB 1 protein. In a preferred embodiment, five amino acids of the MB-1 protein where replaced by other residues.

The tyrosine at position 62, predicted to be buried in the hydrophobic core, was replaced by a tryptophan. This spectral probe has been useful on two counts: it permitted the confirmation of protein purity using fluorescence, and it has been used as a conformational probe. Replacement of Tyr62 by Trp provides increased hydrophobicity to the protein core, and expand the useful spectroscopic properties of the side chain in position 62. Met10, Leu13, Met87 and Leu91 were also replaced to increase the beneficial properties of MB-1 protein. Cysteins were chosen to replace the amino acids found at the 4 latter positions. Preferentially, mutations at position 13 and 87 are performed within a same mutated MB-1 protein while M10C and L91C are found within the same mutated MB-1 protein, said mutated proteins being trivially named MB-1LH and MB-1RH, respectively. Mutating the amino acids is performed using primer having a nuclei acid sequence comprising, SEQ ID NO:11, SEQ ID NO:12, SEQ ID NO:13 SEQ ID NO:14 for mutation of the amino acids 10, 13, 62, 87 and 91, respectively.

The polypeptides as depicted in SEQ ID NO:1, SEQ ID NO:2, SEQ ID NO:3, SEQ ID NO:4, and SEQ ID NO:5 can be separated and purified from a protein occurring in nature. Alternatively, it can be chemically synthesized directly by known methods. It is also possible to prepare the polypeptide of the invention by engineering a gene having a base sequence corresponding to the above polypeptide sequence, inserting the gene into an appropriate expression vector, and expressing the gene in an appropriate host.

In one embodiment of the present invention, there is provided a composition comprising at least one polypeptide having an amino acid sequence selected from the group consisting of SEQ ID NO:1, SEQ ID NO:2, SEQ ID NO:3, SEQ ID NO:4, and SEQ ID NO:5 for endowing a human or an animal with at least one essential amino acid by oral administration.

A further embodiment of the present invention is to provide a polypeptide consisting of an amino acid sequence selected from the group consisting of SEQ ID NO:1, SEQ ID NO:2, SEQ ID NO:3, SEQ ID NO:4 and SEQ ID NO:5, wherein the latter amino acid sequences are encoded by the nucleotide sequences selected from the group consisting of SEQ ID NO:6, SEQ ID NO:7, SEQ ID NO:8 SEQ ID NO:9 and SEQ ID NO:10.

The invention is not limited to these nucleic acid sequences but also extend to all nucleic acid sequences that encodes SEQ ID NO:1, SEQ ID NO:2, SEQ ID NO:3, SEQ ID NO:4 and SEQ ID NO:5. These silent variant sequences include those who comprise nucleic acid changes that alter a specific codon, but where the mutant codon encodes the original amino acid. As well, the invention include nucleic acid sequences that encode for the amino acids originally found in SEQ ID NO:1 to SEQ ID NO:5, but that are no encoded by codons that are considered as universal. This received particular attention for expression of the polypeptide in organelles such as mitochondria or in other organisms species such as Mycoplasma, Tetrahymena or Euplotes. The invention can be adapted for the particular species in which the polypeptide is expressed. Particularly, the nucleic acid sequences can be adapted to the availability of the different tRNA found among species, to provide the preferred codons for a specific amino acid.

The nucleic acid sequences can be cloned into an expression vector designed for production of a recombinant peptide said recombinant peptide being produced in a bacteria, yeast or an eukaryotic cell. As well, the nucleic acid, or the encoded amino acid sequence, can be found as purified molecules or in a transformed cell. It is therefore an embodiment of the present invention to be provided with a cell transformed with an expression vector or any organism or microorganism having integrated a DNA sequence comprising a nucleic acid sequence selected from the group consisting of SEQ ID NO;1, SEQ ID NO:2, SEQ ID NO:3, SEQ ID NO:4 and SEQ ID NO:5.

The present invention will be more readily understood by referring to the following examples which are given to illustrate the invention rather than to limit its scope.

### EXAMPLE I

### Replacement of Tyr-62 by Trp in the Designer Protein Milk Bundle-1 Results in Significant Improvement of Conformational Stability

Materials and Methods

Preparation of MB-1Trp Mutant

Substitution of the Tyr in position 62 by a Trp was performed using the oligo-directed mutagenesis kit <<Altered Sites® II>> (Promega). The mutational oligonucleotide Tyr62-Trp were purified using denaturing polyacrylamide gel electrophoresis (PAGE) and phosphorylated.

| | | |
|---|---|---|
| MB-1: | 5′-ATG GCC ACT ACG TAC TTC | (SEQ ID NO:11) |
| | AAA ACG ATG-3′ | |
| Tyr62Trp: | 5′-ATG GCC ACT ACG TGG TTC | (SEQ ID NO:12) |
| | AAA ACG ATG-3′ | |

The mutation was then confirmed by dideoxynucleotide sequencing using T7 Sequenase™ kit (Amersham Life Science). The mutated MB-1 gene were cloned back in the pCMG20 4-X expression vector and positive clones were checked again by DNA sequencing.

Protein Expression and Purification

Bacteria carrying the mutant vectors were grown at 37° C., 300RPM in 1 L of LB Miller medium (Difco™) to an O.D. of 0.4. Transcription was induced using 1 mM isopropylthio-β-D-galactoside (IPTG) for 3 hours. The cells were then harvested by centrifugation at 3,000×g. The purification procedure was essentially as described bellow. Precipitated cells were resuspended in ice-cold column buffer (10 mM Tris, 200 mM NaCl, 10 mM ethylenediamine-tetraacetate (EDTA), 1 mM sodium azide (NaN₃), pH 7.4). phenylmethylsulfonylfluoride (PMSF), ethylene glycol-bis(β-aminoethylether)-N,N,N′,N′-tetraacetate(EGTA), benzamidine-hydrochloride and benzamide were added to a final concentration of 0.1, 10, 2 and 2 mM, respectively. Cells were then lysed by ten 30-sec sonication pulses using a Branson Sonifier 250 at 60% output control. The sonicated cells were centrifuged at 11,500×g for 30 minutes at 4° C.

The supernatant was then loaded onto a 15 mL amylose column. The maltose binding protein fused with MB-1Trp (MBP-MB-1Trp) was eluted by washing the column with column buffer containing 10 mM maltose (elution buffer). Pooled peak fractions were placed in dialysis tubing (Spectra/Por; MWCO 3,500 Da) with 50 μL factor Xa per 10 mL fusion protein. The bag was placed in 20 mM Tris, 100 mM NaCl, 3 mM CaCl₂ (cleavage buffer) overnight at 4° C. The following morning, the bag was transferred to 10 mM Tris—1 mM EDTA (TE) buffer, pH 8.0. After a 2-hour dialysis, the sample was applied to DEAE-Sepharose equilibrated in TE buffer, pH 8.0 (Fast Flow; Pharmacia) and washed with the same buffer. MB-1Trp was collected as the flow-through. The different fractions were analysed for protein content by the bicinchoninic acid (BCA) assay and the positive fractions were pooled and concentrated using BIOMAX-5K concentrators (Millipore™). Protein samples were prepared in a borate-phosphate buffer (55 mM NaH₂PO₄, 35 mM Na₂B₄O₇, pH 6.8) and dyalised overnight against this buffer prior to measurements. Protein concentration was adjusted to 0.4 mg/mL unless specified otherwise.

Protein Quantification and Electrophoresis

Protein concentration was determined by the BCA assay (Sigma), using bovine serum albumin as the standard. The protein was visualised by sodium dodecyl sulfate polyacrylamide gel electrophoresis (SDS-PAGE) using 12% polyacrylamide-tricine gels, followed by silver nitrate staining. SDS-PAGE experiments were conducted prior to measurements to confirm protein purity.

Conformational Investigation by Circular Dicroism (CD)

Protein samples were degassed for 20 minutes at 20° C. before measurements. Spectra were measured with a Jasco™ J-720 spectropolarimeter, which was routinely calibrated with a 0.06% (W/V) ammonium (+)-10-camphorsulfonate solution. For measurements in the far-UV region a quartz cell with a path length of 0.01 cm was used. Ten scans were accumulated at a scan speed of 20 nm per minute, with data being collected at every nm from 180 to 260 nm. Sample temperature was maintained at 20° C. using a Neslab™ RTE-111 circulating water bath connected to the water-jacketed quartz cuvettes. Spectra were corrected for buffer signal and conversion to As (on the basis of amide bond concentration) was performed with the Jasco™ Standard Analysis software. Secondary structure calculations were performed using the CDsstr program using default settings.

Thermal Denaturation

Samples were prepared as described in the preceeding section. In order to measure thermostability, temperature was increased from 15 to 85° C. at a rate of 30° C. per hour using a Neslab™ RTE-11 controlled by the Jasco™ spectropolarimeter software. CD spectra were collected at every 5° C., from 200 to 260 nm, at a scan speed of 20 nm/min. In order to assess reversibility of thermal denaturation, the protein solutions were cooled down at a rate of 30° C. per hour, and spectra were measured at 70, 50 and 20° C.

Thermal stability was calculated assuming a unimolecular, two-state process as described bellow. The Δε_{MRW} at 222 nm measured at various temperature was used as the property (y) indicative of the extent of unfolding. In the folded state, the parameter y=y_{f} and the fraction of folded protein f_{f} is equal to 1. When the protein is unfolded, the parameter y=yᵤ, and the fraction of unfolded protein fᵤ is equal to 1. For intermediate states, y is given by y_{f}f_{f}+yᵤfᵤ. Thus, by measuring y, we can calculate the fraction of protein unfolded: fᵤ=(y_{f}−y)/(y_{f}−yᵤ). The equilibrium constant for the unfolding process is Kᵤ=fᵤ/(1−fᵤ) and melting temperatures (Tₘ) are obtained at Kᵤ=1.

ANSA Fluorescence Enhancement

Protein concentration was adjusted to 0.1 mg/mL in B/P pH 6.8 and equilibrated at room temperature (RT) for 1 hour. Then ANSA was added to a final concentration of 10 μM and equilibrated 5 minutes prior to measurements. Spectra were recorded using an LS50-B Perkin-Elmer™ fluorometer with an excitation wavelength of 380 nm. Spectra were collected from 410 to 550 nm. Correction for buffer signal on ANSA was keyed in when applicable.

Intrinsic Fluorescence Measurements

Protein concentration of samples was adjusted to 0.1 mg/mL and equilibrated at room temperature (RT) for 1 hour. For chemical denaturation, Urea (Sigma U-5378 ) was added to a final concentration of 8M and equilibrated at RT 5 minutes before measurement. For thermal denaturation, temperature was increased to 85° C. at a rate of 15° C. per hour using Neslab™ RTE-11. Spectra were recorded using an L550-B Perkin-Elmer™ fluorometer with an excitation wavelength of 280 nm. Spectra were then corrected for buffer signal before analysis.

Size-Exclusion Chromatography/Estimation of Molecular Size

Protein samples were adjusted to a concentration of 0.6 mg/mL and loaded onto a Beckmann Ultraspherogel SEC-2000™ column hooked to a Waters 510 HPLC. The column was standardized with the Pharmacia Low Molecular Weight gel filtration calibration standards prepared in the same buffer.

Proteolytic Degradation Measurements

Experiments were carried out as described bellow. Reactions were stopped by the addition of 2% SDS buffer followed by heating for 3 minutes at 100° C. Samples were then analysed on SDS-PAGE followed by silver nitrate staining. The amount of protein remaining after incubation with proteases was determined by measuring the optical density of each band using the image analysis system Imaging Research MCID. Cytochrome c from horse heart (Sigma C-7752) was used as a reference protein for all degradation runs in order to correct for possible variations in crude proteolytic activity.

Results and Discussion

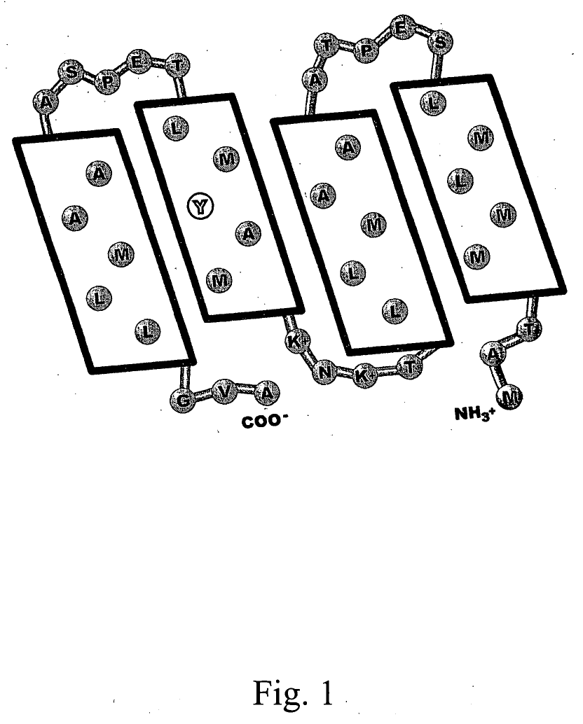

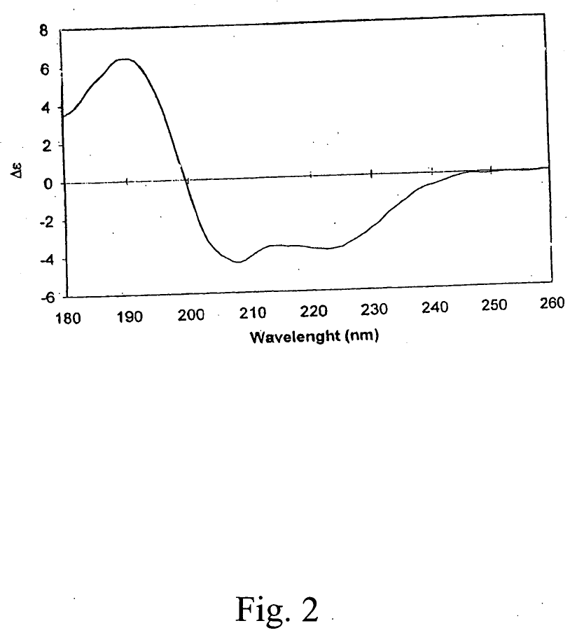

Position 62 in MB-1 was chosen for the emplacement of a spectroscopic probe at the moment of initial design. As shown on the model in FIG. 1, this position is part of the hydrophobic core, and a niche made of 5 Ala was built around it in order to accommodate a larger side chain in this region of the core. Substitution of Tyr62 for Trp was performed on MB-1 by site-directed mutagenesis and confirmed by DNA sequencing and fluorospectroscopy; then the mutant, named MB-1Trp, was characterized. First, circular dichroism (CD) measurements were performed and spectra typical of helical proteins were obtained (FIG. 2). Using CDsstr algorithm, 50% of amide groups were predicted to be in a helical environment. Thus, the secondary structures of the mutant MB-1Trp were found to be similar to the parent molecule MB-1.

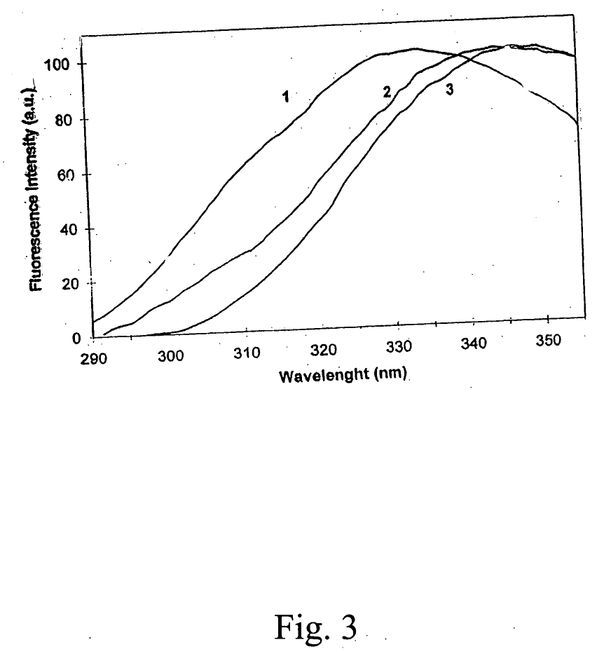

The formation of a tertiary structure in the mutant was confirmed by fluorescence measurements: the folded protein fluorescence peaked at 333 nm, and its emission shifted to approximately 345 nm when unfolded using heat or urea (FIG. 3). The Trp side chain appears to be protected in the folded protein, and exposed to solvent upon unfolding, as predicted by design. Similar shifts in fluorescence λₘₐₓ were observed for another designer protein (α4) after insertion of a Trp side chain.

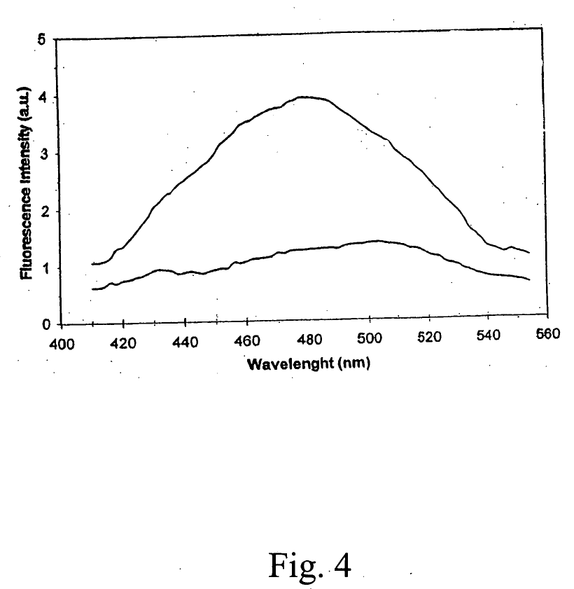

Results from fluorescence measurements using the probe ANSA are shown in FIG. 4. The enhancement of ANSA emission by the proteins was limited to a factor of 3, which is comparable to the parent molecule, and lower than the values expected for poorly folded proteins. These results suggest that MB-1Trp is rather well folded, and not in a molten globule state. Size-exclusion chromatography analysis revealed that MB-1Trp migrated as a 12 kDa protein, closed to the expected size of an MB-1Trp monomer. Substitution of Trp in position 62 appears to correct one weakness of the initial design of MB-1, which had a size approaching that of a dimer.

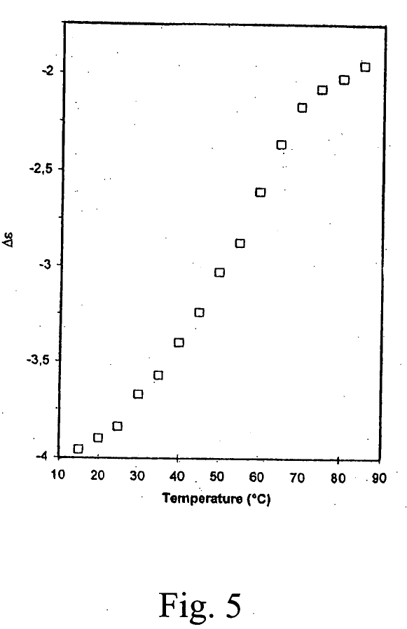

The impact of the substitution in position 62 on conformational stability was verified using two different approaches. First, the CD signal at 222 nm was recorded at various temperatures in order to monitor unfolding of MB-1Trp helices. The denaturation of MB-1Trp (FIG. 5) indicated a melting temperature of 55° C., a significant improvement over MB-1 (melting temperature of 39° C.) and other mutants characterized so far. The thermal denaturation was found to be fully reversible, another improvement over MB-1, but the transition was spread over a wide temperature range (30° C. to 65° C.). However, this apparent lack of cooperation in MB-1Trp thermal unfolding also characterized MB-1 and other small designer proteins.

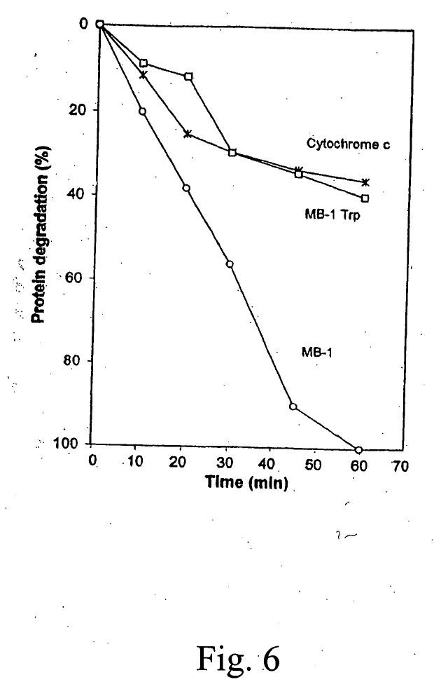

Stabilization of MB-1 fold by the mutation was also confirmed by proteolytic degradation experiments. Degradation curves shown in FIG. 6 clearly demonstrate a gain in resistance to proteolytic attack. Under standard conditions, MB-1Trp was twice as resistant as MB-1, and behaved like a natural protein of similar size (cytochrome C).

Trp and Tyr residues are comparable on various counts: they have large aromatic side chains, they undergo a limited but similar loss of conformational entropy upon protein folding; and they have similar secondary structure propensities. They differ in their hydrophobicities however, due to the presence of a hydroxyl group in p position on the phenyl moiety. When such a polar group is involved in a hydrogen bond, the difference between Trp and Tyr hydrophobicity decreases, but in MB-1, no such H-bonding partner was properly positioned while designing the protein. Most results shown here indicate that the increase in hydrophobicity in position 62, due to the removal of tyrosine's hydroxyl group from the core, led to an important improvement of MB-1 fold stability. This may be explained by tryptophan's ability to make extensive van der Waals contacts with neighboring residues due to its large size, in addition to its contribution to the hydrophobic effect. The improvement of MB-1 properties also included the specification of a monomeric organization.

Trp and Tyr both belong to the class of large side chains, but Trp is significantly larger than Tyr, with an additional 34 cubic Angstroms. The original design strategy allowed for a Tyr in position 62, but not for Trp, which would result in a layer volume that would be above average for natural bundles. The results shown here all indicate that MB-1 secondary and tertiary structures were not disturbed by the substitution Tyr62-Trp. The protein is helical and well folded and it appears that the niche around position 62 readily accommodates the larger side chain of Trp. The mutation resulted in an important improvement in the protein stability

The gain in conformational stability afforded by the Tyr62-Trp mutation is of paramount importance for future advances in the development of MB-1 family of proteins. Its high resistance to proteases permit the production of this high quality protein by transgenic crops to be used in animal production. A comparison of previously reported analyses of plant protein degradability indicates that MB-1Trp compares to sunflower 2S seed albumin 8 protein, a protein with a high methionine content intended for production of transgenic crops with enhanced nutritional quality. Note that the EAA profile of SFA8 is not optimized for lactating cows needs, while MB-1Trp is, due to its balanced content of methionine, lysine and threonine. This comparison indicates that although few cycles of design may be required, our design approach produced a high quality protein that competes with natural proteins at the level of stability.

The mutant presented here clearly outperforms the parent molecule on several counts, including the ability to stay monomeric under conditions used here, and to resist degradation in solution.

### EXAMPLE II

### Engineering Nutritious Proteins: Improvement of Stability in the Designer Protein MB-1 via Introduction of Disulfide Bridges

Materials and Methods

Preparation of the New Mutants

Substitution of Cys in position 13 and 87 of MB-1 (MB-1LH) and positions 10 and 91 of MB-1 (MB-1RH) was performed using the oligo-directed mutagenesis kit <<Altered Sites® II>> (Promega). The mutational oligonucleotides (1 for position 13; 2 for position 87, 3 for position 10; 4 for position 91 shown below with the corresponding MB-1 sequences) were purchased from GibcoBRL/Life Technologies™, purified using denaturing polyacrylamide gel electrophoresis (PAGE) and phosphorylated.

| | | |
|---|---|---|
| MB-1: | 5′-ATG ATG ACC ACC CTG TTT | (SEQ ID NO:13) |
| | AAA ACT ATG-3′ | |
| Oligo 1: | | |
| L13C: | 5′-ATG ATG ACC ACC TGC TTT | (SEQ ID NO:14) |
| | AAA ACT ATG-3′ | |
| MB-1: | 5′-ACG GCT ACA ACC ATG AAA | (SEQ ID NO:15) |
| | AAT CAT CTG-3′ | |
| Oligo 2: | | |
| M87C: | 5′-ACG GCT ACA ACC TGC AAA | (SEQ ID NO:16) |
| | AAT CAT CTG-3′ | |
| MB-1: | 5′-ATG ACC GAC ATG ATG ACC | (SEQ ID NO:17) |
| | ACC CTG TTT-3′ | |
| Oligo 3: | | |
| M10C: | 5′-ATG ACC GAC ATG TGT ACC | (SEQ ID NO:18) |
| | ACC CTG TTT-3′ | |
| MB-1: | 5′-ATG AAA AAT CAT CTG CAG | (SEQ ID NO:19) |
| | AAC TTG ATG-3′ | |
| Oligo 4: | | |
| L91C: | 5′-ATG AAA AAT CAT TGC CAG | (SEQ ID NO:20) |
| | AAC TTG ATG-3′ | |
| MB-1: | 5′-ATG GCC ACT ACG TAC TTC | (SEQ ID NO:21) |
| | AAA ACG-3′ | |
| Oligo 5: | | |
| Y62W: | 5′-ATG GCC ACT ACG TGG TTC | (SEQ ID NO:22) |
| | AAA ACG-3′ | |

The Tyr62-Trp mutation lead to MB-1Trp and was achieved directly in the expression vector pMal-c2. The mutational oligonucleotide 5 was used and treated as described above. The mutations were then confirmed by dideoxynucleotide sequencing using T7 Sequenase kit (Amersham Life Science). The mutated MB-1 genes were cloned back in pCMG20 4-X, the expression vector. All positive clones were checked again by DNA sequencing after cloning.

All mutated proteins were prepared as described in Example I. After purification, all proteins were checked for purity by sodium dodecyl sulfate polyacrylamide gel electrophoresis (SDS-PAGE) (protein purity >95%).

Protein concentration was determined by the bicinchoninic acid (BCA) assay (Sigma), using bovine serum albumin as standard. The protein was visualised by sodium dodecyl sulfate polyacrylamide gel electrophoresis (SDS-PAGE) using 12% polyacrylamide-tricine gels, followed by silver nitrate staining. SDS-PAGE experiments were conducted prior to measurements to confirm protein purity.

Quantification of Cys Residues and Assessment of Bridge Formation

Chemical modification of the Cys residues by iodoacetamide and iodoacetate was performed as described bellow. Comparison of the electrophoretic mobility of the reduced and non-reduced form of the MB-1 mutant was done by SDS-PAGE. The Cys were also quantified by reacting with p-hydroxymercurybenzoate (p-HMB) and measuring absorbance of p-HMB at 260 nm with a UV/vis spectrophtometer (Novaspec™, Pharmacia).

Conformational Investigation by Circular Dichroism (CD)

Unless specified otherwise, protein samples were prepared at a concentration of 0.6 mg/mL with a phosphate buffer (128 mM NaH₂PO₄, pH 6.8). The reductive agent dithiothreitol (DTT) was added to a final concentration of 10 mM where specified. The samples were then degassed and equilibrated 20 minutes at 20° C. before measurements. Spectra were measured with a Jasco™ J-720 spectropolarimeter, which was routinely calibrated with a 0.06% (w/v) ammonium (+)-10-camphorsulfonate solution. For measurements in the far-UV region, a quartz cell with a path length of 0.01 cm was used. Ten scans were accumulated at a scan speed of 20 nm per minutes, with data being collected at every nm from 180 to 260 nm. Sample temperature was maintained at 20° C. using a Neslab™ RTE-111 circulating water bath connected to the water-jacketed quartz cuvette. Spectra were corrected for buffer signal and conversion to Δε_{MRW} (mean residue weight) was performed with the Jasco Standard Analysis software. Secondary structure calculations were performed using the CDsstr program using default settings.

Thermal Denaturation

Samples were prepared as described in the preceding section. In order to measure thermostability, temperature was increased from 10 to 95° C. at a rate of 30° C. per hour using a Neslab™ RTE-11 controlled by the Jasco spectropolarimeter software. CD spectra were collected at every 5° C., from 200 to 260 nm, at a scan speed of 20 nm/min and CD signal at 222 nm were collected at 1° C. interval. In order to assess reversibility of thermal denaturation, the protein solutions were cooled down at a rate of 30° C. per hour, and spectra were measured at 70, 50 and 20° C.

Thermal stability was calculated assuming a unimolecular, two-state process as previously described. The CD signal at 222 nm at various temperature was used as the property (y) indicative of the extent of unfolding. In the folded state, the parameter y=y_{f} and the fraction of folded protein f_{f} is equal to 1. When the protein is unfolded, the parameter y=yᵤ, and the fraction of unfolded protein fᵤ is equal to 1. For intermediate states, y is given by y_{f}f_{f}+yᵤfᵤ. Thus, by measuring y, we can calculate the fraction of protein unfolded: fᵤ=(y_{f}−y)/(y_{f}−yᵤ). The equilibrium constant for the unfolding process is Kᵤ=fᵤ/(1−fᵤ) and melting temperatures (Tₘ) are obtained at Kᵤ=1.

8-anilinonaphthalenesulfonic acid (ANSA) Fluorescence Enhancement

Protein concentration was adjusted to 0.2 mg/mL and equilibrated at room temperature (RT) for 1 hour. Then ANSA was added to a final concentration of 10 μM and equilibrated 5 minutes prior to measurements. Spectra were recorded using an LS50-B Perkin-Elmer™ fluorometer with an excitation wavelength of 380 nm. Spectra were collected from 410 to 550 nm. Correction for buffer signal and for the effect of DTT on ANSA was keyed in when applicable.

Fluorescence Quenching Measurements

Protein concentration of samples was adjusted to 0.2 mg/mL and equilibrated at RT for 1 hour. Where specified DTT was added to a final concentration of 10 mM and equilibrated at RT 15 minutes before adding the quencher citrate (final concentration 0.25 M). Control samples were exposed to 0.75 M NaCl in order to keep the same ionic strength as in the samples exposed to 0.25 M citrate. Mutants containing Tyr were excited at 280 nm and fluorescence spectra were recorded from 290 to 330 run. For the Trp mutants, fluorescence was collected from 310-400 nm. Quenching of the Trp mutants was performed with 0.3M potassium iodate, and NaCl was used in controls. Spectra were then corrected for buffer signal before analysis. The effect of DTT on fluorescence was corrected when needed.

Proteolytic Degradation Measurements

Proteins were dialysed against 2000 volumes of borate-phosphate buffer (pH 6.8) at 4° C. overnight. Experiments were then carried out as described bellow. Reactions were stopped by the addition of 2% SDS buffer followed by heating for 3 minutes at 100° C. Samples were then analysed on SDS-PAGE followed by silver nitrate staining. The amount of protein remaining after incubation with proteases was determined by measuring the optical density of each band using the image analysis system Imaging Research MCID. Cytochrome C (horse heart, Sigma) was used as a reference protein for all degradation runs in order to correct for possible variations in crude proteolytic activity.

Size Exclusion Chromatography (SEC)

Proteins were applied to a Beckman Ultraspherogel™ SEC-200 column at a concentration of 0.6 mg/mL. A constant flow of 0.8 mL/min was maintained using a Waters HPLC pump. Pharmacia Low Molecular Weight gel filtration calibration standards were used for column calibration.

Results and Discussion

Design Strategy

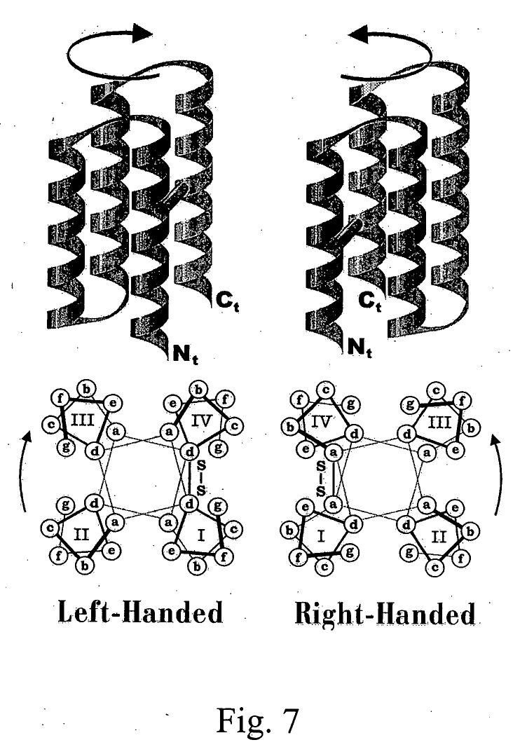

The putative modifications to MB-1 structure are illustrated in FIG. 7. The design strategy used here focused on two aspects: 1-the restrictive effect of a covalent bond between remote residues on the protein as a whole; and 2-the precise location of Cys which permits disulfide bridge formation. By choosing positions as far apart as possible, one can reduce the entropy gain upon unfolding for most of the protein. Thus, insertion of a bridge between helices I and IV would enclose a larger part of the polypolypeptide than a bridge involving other helices. Another consideration for using helix I is that this MB-1 segment of sequence is sensitive to proteolytic degradation. The restriction of helix I by Cys insertion could help prevent such a phenomenon.

The position of Cys in helices I and IV must allow sulfydryl groups to be properly aligned in order to minimise strain induced by bridge formation. On the basis of geometric models built for similar proteins, it appeared that position “d” of the heptad pattern used for MB-1 design would offer the best geometry for bridge formation. Therefore, L13 and M87 residues were selected for mutation to Cys. FIG. 7 depicts the expected location of the bridge in the mutant (hereafter referred to as MB-1LH, assuming it folds as per design). Note that for proper alignment of position “d” in helices I and IV, a left-hand connectivity of the helices had to be assumed (i.e. the bundles are positioned such that when helix I is at the fore front, with its N-terminus pointing down, then helix II is placed to the left of helix I). A second scenario was considered, in which a right-hand connectivity could be specified. Examination of the second model in FIG. 7 suggests mutations at positions “a” in helices I and IV, since positions “d” would be too far apart. By choosing M10 and L91 residues for mutation to Cys, we attempted to generate a mutant (named MB-1RH) that would resemble MB-1LH as much as possible, except for reversing its connectivity.

Disulfide bridges was also inserted into MB-1Trp. This protein is a derivative of MB-1 where Tyr62 was replaced by Trp. Position 62 in MB-1 was chosen for the emplacement of a spectroscopic probe at the moment of initial design. As shown on the model in FIG. 1, position 62 is part of the hydrophobic core, and a niche made of 5 Ala was built around it in order to accommodate a larger side chain in this region of the core. The replacement of Tyr by Trp was thought to improve on stability, and indeed, characterisation of MB-1Trp confirmed the strategy. MB-1Trp has a melting temperature of 55° C. and is more resistant to protease action than MB-1. Here we are going to use MB-1Trp because of the increase in bulk offered by Trp in the core, in a way to compensate for the loss of volume consequent to the mutations used for bridge insertion.

Initial Characterisation of Mutant Structure

The presence of two Cys in all mutants was confirmed by Cys derivatization with mixtures of iodoacetamide and iodoacetate, and by reacting Cys with p-HMB, in agreement with the DNA sequences of the expression vectors. Formation of the bridges was confirmed by a comparison of the protein migration after and before treatment with DTT, a reductive agent. Reduction of the protein changed its migration speed, and the impact of reduction appeared to modify mobility of all proteins treated. Replacement of Tyr by Trp was confirmed by spectrofluorometry.

Secondary Structures Analysis

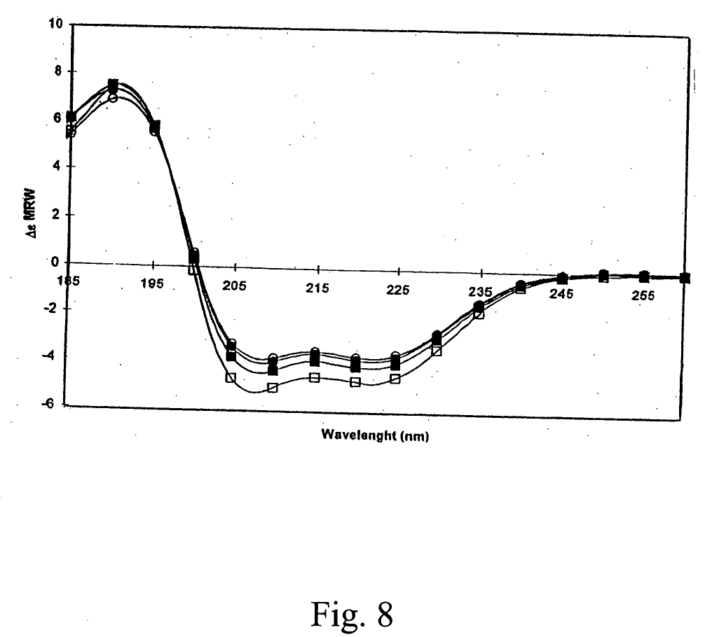

Secondary structure analysis of the mutant was performed using circular dichroism (FIG. 8, Symbols are: open circles for MB-1LH; open squares for MB-1RH; closed circles for MB-1TrpLH; and closed squares for MB-1TrpRh). The spectra measured for all mutants with their bridge closed (no DTT) were typical of helical proteins, and calculations of helical amide contents were in the range expected for these proteins (Table 1). MB-1LH, MB-1RH and MB-1TrpLH appeared to have similar secondary structures as their parent molecules MB-1 and MB-1Trp (which contain approximately 50% helical amides. The protein MB-1 appeared to be tolerant to bridge insertion, regardless of the connectivity imposed by this bridge (either handedness).

Inserting the bridges leading to a right-hand connectivity into MB-1Trp led to a different scenario: the helical content of MB-1TrpRH differed significantly in secondary structure contents, with only 42% helical content (Table 1). The right-hand connectivity in MB-1TrpRH imposes a different conformation, with less helical content.

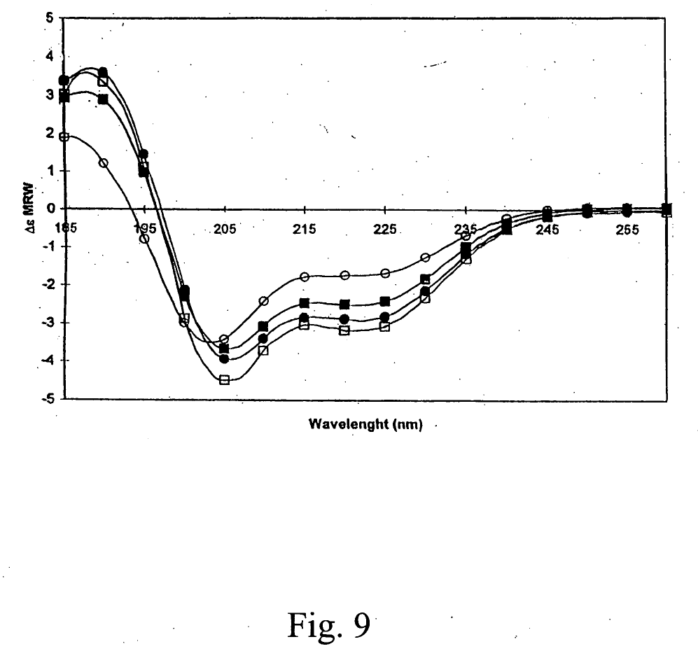

The mutants were then analysed in the presence of DTT (FIG. 9, Symbols are: open circles for MB-1LH; open squares for MB-1RH; closed circles for MB-1TrpLH; and closed squares for MB-1TrpRh), in order to separately assess the effect of the mutations (insertion of Cys) from the impact of bridge formation. All mutants helical contents dropped when exposed to DTT as indicated by a loss of signal intensity at 190 and 225 nm, with MB-1LH being nearly completely unfolded (Table 1). This suggests that in the absence of the bridge, mutations used here are destabilising.

TABLE 1
Comparison of physical properties measured for the four mutants

| | | Monomer | | | |
|---|---|---|---|---|---|
| | | population | Monomer population | | Protein |
| Mutant | α-helix^{a} +/− 3% | SEC^{b} +/− 5% | SDS-PAGE^{c} +/− 5% | Tm^{d} +/− 1° C. | undegraded^{e} +/− 8% |
| RH | 50 | 100 | 100 | 50 | 35 |
| | (23)^{f} | | | | |
| LH | 48 | 100 | 100 | 48 | 43 |
| | (18) | | | | |
| MB-1TrpRH | 42 | 90 | 95 | 49 | 40 |
| | (25) | | | | |
| MB-1TrpLH | 55 | 100 | 100 | 42 | 17 |
| | (34) | | | | |

^{a}Helix percentage calculated with CD spectra.
^{b}Oligomerization population estimated from exclusion chromatography.
^{c}Oligomerization population estimated from SDS-PAGE gel analysis
^{d}Melting temperature derived from thermal denaturation.
^{e}Percentage of protein undegraded after a 60 minutes of incubation with pronase E.
^{f}Numbers in parenthesis are for proteins with opened disulphide bridge (treated with DTT).

Among the four mutants presented here, MB-1LH had the largest drop in helical content after bridge opening (down to 18%, see Table 1). This means that the mutations M13-Cys and L87-Cys promote unfolding of the protein, and that formation of the bridge compensates for this deleterious effect.

When the same treatment was performed on MB-1TrpLH, the loss of helical content was less severe (down to 34%) (Table 1). The fact that MB-1TrpLH stay somewhat better folded (34%) than MB-1LH in the presence of DTT may suggest that Trp compensate for the loss of bulk consequent to Cys insertion in the core with a left-hand connectivity. Partial unfolding after DTT treatment was confirmed by fluorescence quenching and ANSA binding for all mutants.

Reversibility of DTT-induced unfolding was verified by CD measurements. After DTT removal, CD spectra were similar to the native spectra, suggesting that all four proteins refolded after bridge repair (FIG. 8).

Quaternary Structure

The ability to control protein association and aggregation has been (and still is) an important tumble stone in de novo protein design. The protein association was monitored for the mutants under two sets of conditions. First, quaternary organisation was monitored by SEC under benign buffer conditions in a way to observe the impact of both covalent and non-covalent bonds. Then, the proteins were denatured and migrated on an SDS-PAGE gel in order to detect any inter-molecular covalent bond. Results in Table 1 indicate that all mutants, except MB-1TrpRH, were monomeric, regardless of the treatments used here. This confirms that intra-molecular bridges are formed, and suggest that the three proteins fold as planned. At variance, MB-1Trp RH was found to contain 5-10% dimer that resisted SDS treatment, which indicate that these dimers are formed via an inter-molecular bridges. Other oligomerization states have been observed for this protein under specific experimental conditions. An increase in surface hydrophobicity was detected using ANSA binding measurements (ratio I₄₈₀/I₅₁₀ for MB-1TrpRH=12, compared to a ratio of 7-8 for the other three mutants), which indicate that MB-1TrpRH core is more fluid, allowing for inter-molecular bridges.

Effect of the Mutations on Conformational Stability

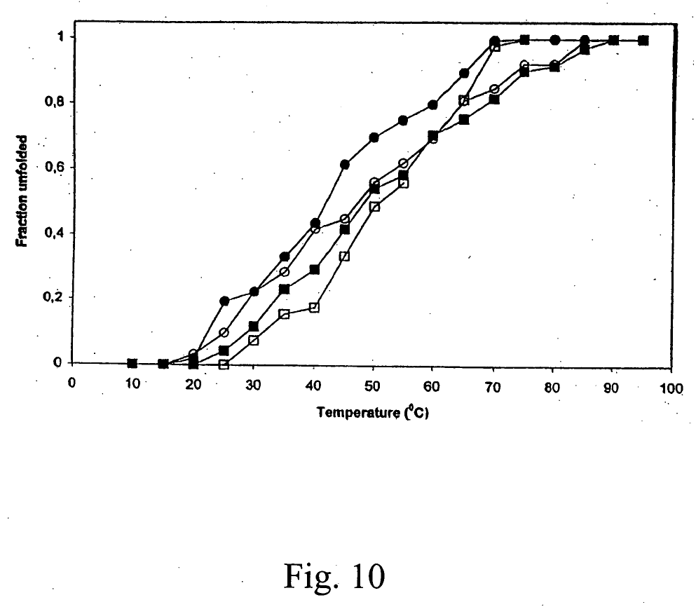

Conformational stability of both mutants was measured using CD as described in Materials and Methods. FIG. 10 shows the four denaturation curves, while calculated Tm values are listed in Table 1. Both MB-1 mutants clearly outperform MB-1 (Tm=39° C. (24)), by approximately 10° C. in thermostability. Symbols are: open circles for MB-1LH; open squares for MB-1RH; closed circles for MB-1TrpLH; and closed squares for MB-1TrpRh. Approximately 100% of the helical content was recovered after denaturing the protein by cooling to room temperature as described earlier, whereas the MB-1 parent molecule could not be refolded under similar experimental conditions.

Insertion of bridges into MB-1Trp resulted in a completely different condition, both mutants being less stable than their ancestor molecule MB-1Trp (Tm=55° C.), with MB-1TrpLH being the least stable (Tm=42° C.). The mutants seemed less stable than the pair MB-1LH and RH.

Effect of the Mutations on Proteolytic Degradation

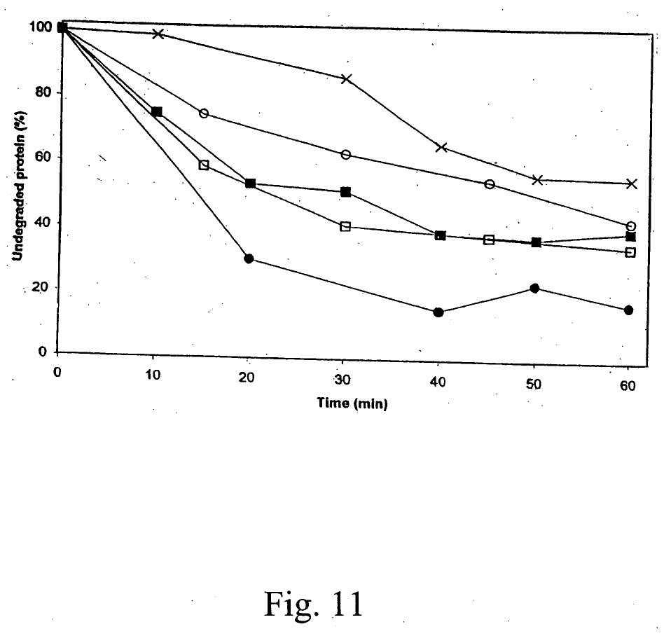

Proteolytic degradation experiments were carried out as described in FIG. 11. Symbols are: open circles for MB-1LH; open squares for MB-1RH; closed circles for MB-1TrpLH; and closed squares for MB-1TrpRh. Both mutants derived from MB-1 outperformed MB-1, with about 40% intact protein left after a 1 hour treatment with proteases (MB-1 cannot be detected after same treatment). MB-1LH is more resistant than MB-1RH in the first 45 min, but the difference decreases after 60 min. The mutants derived from MB-1Trp were less stable than their parent molecule (60% intact MB-1Trp left under same conditions). MB-1TrpRH behaved like MB-1RH and the other MB-1 mutants, while MB-1TrpLH has the lowest resistance, with 17% proteins left after treatment (Table 1). Once again, the insertion of Trp near the bridge did not lead to any improvement on stability. Degradations were performed in the presence of DTT and higher degradation rates were measured. However, difficulties were encountered due to the inhibitory effect of DTT on the proteolytic activity and the need to prevent re-oxidation of the disulfide bridge, thus strongly limiting the reliability of these data.

The insertion of disulfide bridges in MB-1 was found to have an important impact on protein behaviour. Both mutant proteins were more resistant and stable than MB-1. Their unfolding (be it after bridge reduction or due to high temperature) was found to be reversible, and their apparent size in solution indicates they are monomeric. Both strategies led to serious behaviour improvement when compared to the MB-1 parent molecule, in accordance to the impact of disulphides on protein resistance. This phenomenon cannot be ascribed to Cys mutations per se (which are destabilising), but is a consequence of specifying connectivity and limiting protein skeleton freedom through disulfide bond formation.

Predicting (and understanding) the impact of Cys mutations separately from the effect of bridge formation have been difficult in the past. As a result, engineering disulfides in natural proteins have sometimes led to loss of stability. Separately monitoring the effect of a mutation from that of bridge formation was performed. Results obtained in the presence of DTT indicate that Cys mutations are destabilizing for both proteins. A negative impact of Cys insertion in heptad positions “a” and “d” may be observed. Cys is a poor helix former, it is smaller than Met or Leu, and its polarity is higher than that of Met or Leu.

Closing the bridge led to an increase in stability, which suggests that possible strain induced by cross-linking helix I and IV may be less important. Thus, the geometry and the position of the Cys residues appear to be adequate for bridge formation as expected per design.

In MB-1 Trp, the core Tyr62 has been replaced by Trp, leading to an increase in hydrophobicity and bulk in the core. Thus, we attempted to compensate the impact of the Cys mutations by using MB-1Trp for target. The disulfide bridged constructions harbouring Trp 62 analysed here were not better behaved than the ones based on MB-1. MB-1TrpRH appeared to form concatemers linked by inter-molecular bridges, indicating a high conformational flexibility. MB-1TrpLH had the lowest resistance to proteases, suggesting some undetected flaw in folding. Therefore, the insertion of Trp in position 62 of bridged MB-1 protein is not appropriate, and one might infer that this position is too close to the bridges, promoting steric hindrance.

The mutations used here allowed to increase MB-1 stability and resistance to proteases, which improves MB-1 analogs or mutants efficiency as a food additive.

### EXAMPLE III

### Controlled Polymerization of the Synthetic Polypeptide MB-1TrpRH

Formation of inter-molecular disulfide bridges between protein monomers may allow to expand the range of applications for such a protein. Polymerization contributes to protein stability. The proximity of proteins attached into a polymer excludes large proteases and allow for protection of otherwise exposed targets. Polymerisation would thus improve on production yield in a given in vivo environment (bacteria or plants) and improve on the performances of the polypeptide when used as a feed additive in polygastric animals such as cows. Polymerising proteins may also be of interest in molecular biology diagnostics. For example, SDS-PAGE protein standards are often made of a mixture of different proteins, with different staining properties.

As a results, stain density may vary greatly from one protein to the next. Further, most protein standard kits include proteins with size that may not cover the range of sizes to be measured in a regular manner. A solution to these limitations is a ladder made of similar proteins, separated by a fixed increment when going from a band to the other. Such a ladder is actually available on the market, and is made of proteins obtained from genetic constructions where an increasing number of coding regions are fused (BioRad™, Precision Protein Standard) in one gene. In this kit, proteins of different sizes are produced by different transformed microbes producing monomer, dimers, trimers, and higher oligomers of the same subunit (monomer), extracted and mixed in proper amounts in order to serve as molecular weight markers. Since one transformed organism encodes only one association state (i.e. one clone produces dimers, the other one expresses trimers, etc.), a number of organisms have to be maintained, grown, harvested in order to produce protein of different sizes. Here we report a protein ladder produced by one transformed organism, which may simplify the preparation of protein molecular weight markers.

Materials and Methods

The protein used as the monomer (or repeating unit) is a mutant of the designer protein MB-1 where the mutations Met10-Cys, Leu91-Cys and Tyr62-Trp were performed by oligonucleotide site-directed mutagenesis. The production by Escherichia coli and purification protocol is as reported in Morrison et al. 2000, except for few modifications. A salt gradient was used (0 to 50 mM NaCl) in order to elute the proteins from a DEAE Sepharose column. Fraction containing the mutant MB-1TrpRH were collected and concentrated using Millipore concentrators. The proteins were then incubated in 10 mM DTT for 8 h at 4° C. The reducing agent was removed by dialysis against 100 volumes of borate phosphate buffer pH 7.6 for 12 h at 4° C. After this cycle of reduction/oxidation of the disulfide bridges, the proteins solution was loaded onto a G-50 Sephadex™ column and fractions were analysed for their protein contents by SDS-PAGE. A 10% tricine buffer was used and the gels were stained using silver staining.

Results and Discussion

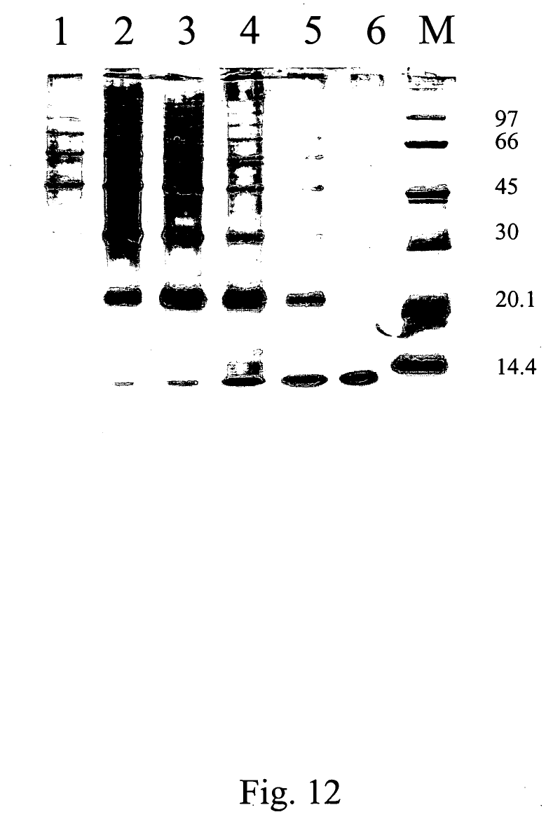

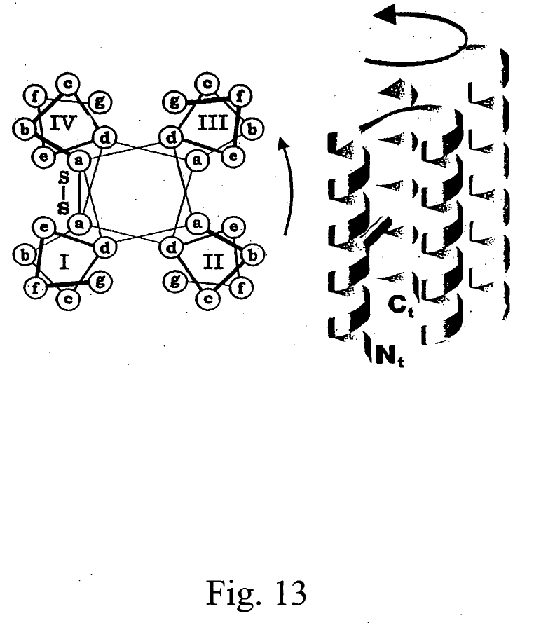

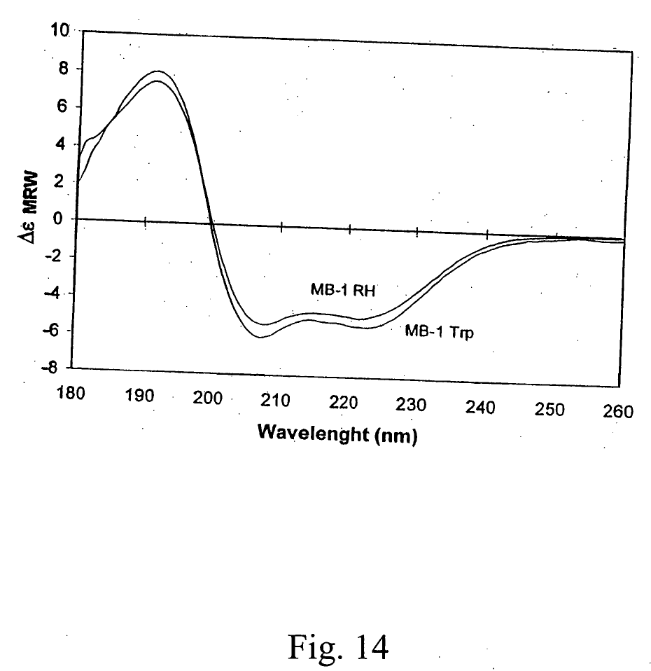

The present experiment illustrates a number of proteins intended to fold into cc-helical proteins. One of the mutant we obtained, named MB-1Trp RH, is able to form intra-and inter-molecular disulfide bridges. Interestingly, the association of the mutant MB-1TrpRH is dependant on the way the oxidation of the bridges is carried out. The treatment described in Methods led to the formation of oligomers, with a rather even distribution of sizes in the range 11-125 kDa. The electrophoretic pattern of fractions collected from the size exclusion column is shown in FIG. 12. The fractions 5 and 6 contained mainly monomeric and dimeric MB-1TrpRH, migrating at 12 and 21 kDa respectively. The other lanes contained a number of oligomer proteins with the following estimated sizes: 12, 21, 35, 50, 60, 71, 79, 87 kDa, and other proteins of larger sizes (up to 125 kDa) that cannot be estimated with the gel system and protein markers used here. All these bands are in fact oligomers of MB-1TrpRH, a protein of 11.3 kDa. Circular dichroism (CD) measurements were performed and spectra typical of helical proteins were obtained (FIG. 13). Addition of DTT eliminated dimers and higher oligomers, which converted into monomers (FIG. 14).

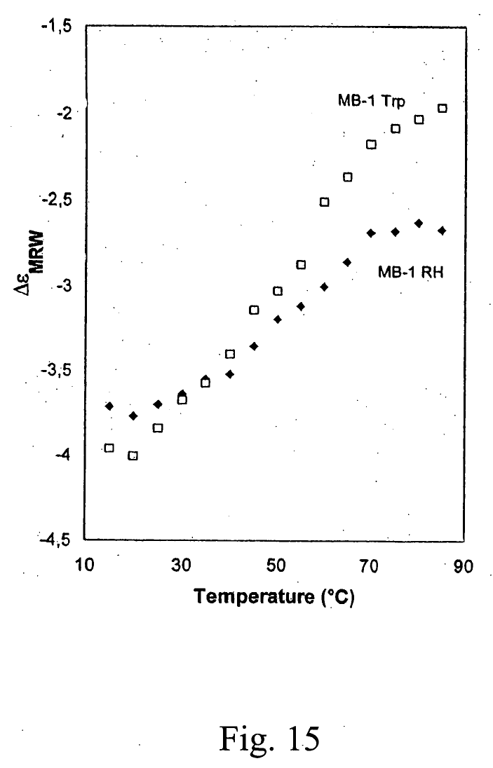

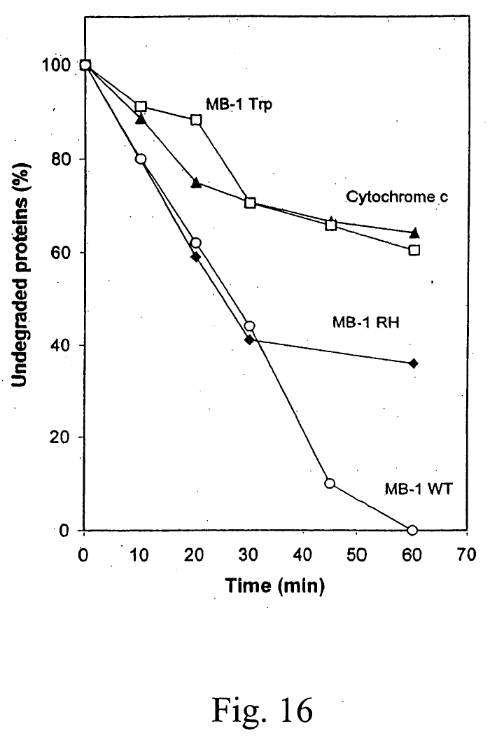

In the present experiment, MB-1TrpRH has 2 cysteines, therefore, one intra-molecular bridge may form in a monomer (FIG. 15). The protein has to form bridges with other monomers (i.e. the bridges cannot be intra-molecular) in order to form dimers or higher oligomers. Since those bridges involve Cys side chains that are expected to be in core positions in the monomers, it appears that formation of the ladder involve a drastic change in conformation for MB-1TrpRH subunits in dimer and higher oligomers. Interestingly, the proteins stay soluble, even after extensive cross-linking, which indicate that somehow, the core side chains are protected in the new cross-linked conformation. Degradation curves shown in FIG. 16 clearly demonstrate a gain in resistance to proteolytic attack.

Here the synthesis of various oligomers of a mutant proteins produced by a single bacterial clone was shown. After a cycle of reduction/oxidation of the disulfide bridges, the oligomer range in sizes from 11 kDa to approximately 125 kDa, and migrate as a ladder on a SDS-PAGE gel. Such a ladder is convenient in that it is made of the same proteins with size that are multiples of 11 kDa.

### EXAMPLE IV

### Point Mutation of Surface Amino Acids of MB-Trp-1

Materials and Methods

Preparation of the New Mutants.

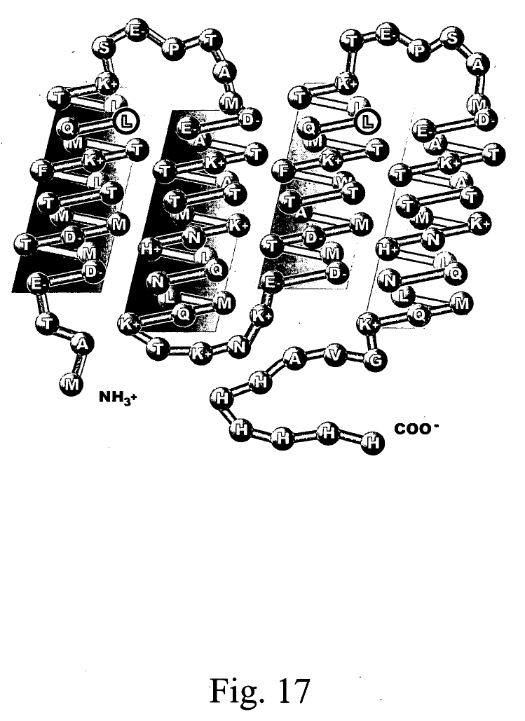

Substitution of leucines in position 19 and 68 by glutamate on the template protein MB-1Trp (FIG. 17) was performed using “Altered Sites II mutagenesis kit” (Promega) and “Site-directed Mutagenesis kit” (Stratagene), respectively. The mutagenic oligonucleotides are shown below with the corresponding MB-1Trp sequence (oligo 6 for position 19 and oligo 7 and 8 for position 68). They were purchased from Invitrogen Life Technologies, purified using polyacrylamide gel electrophoresis (PAGE) and phosphorylated.

| | | |
|---|---|---|
| MB-1Trp: | 5′-TTT AAA ACT ATG CAG CTG | (SEQ ID NO:23) |
| | TTG ACC AAG TCG -3′ | |
| Oligo 6: | | |
| L19E | 5′-TTT AAA ACT ATG CAG GAA | (SEQ ID NO: 24) |
| | TTG ACC AAG TCG -3′ | |
| MB-1Trp: | 5′-CGTGG TTC AAA ACG ATG | (SEQ ID NO:25) |
| | CAG TTG TTA ACG AAG ACC GAG | |
| | CCCTC-3′ | |
| Oligo 7: | | |
| L68E: | 5′-CGTGG TTC AAA ACG ATG | (SEQ ID NO:26) |
| | CAG GAG TTA ACG AAG ACC GAG | |
| | CCCTC-3′ | |

and the reverse complement of Oligo2

| | | |
|---|---|---|
| Oligo 8: | | |
| L68E: | 5′-GAGGG CTC GGT CTT CGT | (SEQ ID NO:27) |
| | TAA CTC CTG CAT CGT TTT GAA | |
| | CCACG -3′ | |

The mutations were confirmed by dideoxynucleotide DNA sequencing. The combination of both mutations was performed by applying the same method as the one used for Leu68Glu substitution, but on the template MB-1Tyr62Trp/Leu19Glu. The mutated genes were expressed in pMal-c2 expression vector (New England Biolabs) in fusion with the MalE gene. The poly-histidine tag (His-tag) was ligated afterward to the gene by constructing a chimeric plasmid from the original pMal-c2 MB-1Trp and pMal-c2 MB-1His, which is a similar plasmid containing MB-1 gene with a His-tag at the C-terminal. All constructions were confirmed by dideoxynucleotide DNA sequencing.

Protein Expression and Purification

All mutants were expressed and purified as described previously in Example I, with the following modifications. The secondary chromatography involved metal affinity purification, as described in Grundy and coworkers (Grundy et al., 1998, Prot. Expr. Purif., 13:61-66). However, ethylenediaminetetraacetate (EDTA) and ethylene glycol-bis(β-aminoethylether)-N,N,N′,N′-tetraacetate (EGTA) were omitted from the protease inhibitor cocktail to avoid column striping.

Protein Quantification and Electrophoresis

Protein concentration was determined by bicinchoninic acid (BCA) assay (Sigma) using bovine serum albumin as standard. The proteins were visualized by SDS-PAGE using 16% acrylamide-tricine gels and silver staining (Amersham Biosciences) prior to measurements to confirm protein purity.

Conformationnal Investigation by Circular Dichroism (CD)

Unless specified otherwise, experiments were conducted at a protein concentration of 0.6 mg/ml with a phosphate buffer (128 mM NaH2PO4, pH6.8). CD measurements were carried out as described previously in Example I.

Thermal Denaturation

The samples were prepared as described in the preceding section. To measure thermostability, temperature was increased from 5 to 98° C. at a rate of 20° C./h using a Neslab RTE-111 water bath controlled by the Jasco™ spectropolarimeter software. CD spectra were collected by a Jasco™ spectropolarimeter J-720 at every 5° C. from 190 to 260 nm, at a speed of 20 nm/min, and CD signals at 222 nm were collected at every 1° C.

Thermal stability was calculated assuming a unimolecular, two-state process as previously described in Example I. The CD signal at 222 nm at various temperature was used as the property (y) indicative of the extent of unfolding. In the folded state, the parameter y=y_{f} and the fraction of folded protein f_{f} is equal to 1. When the protein is unfolded, the parameter y =yᵤ, and the fraction of unfolded protein fu is equal to 1. For intermediate states, y is given by y_{f}f_{f}+yᵤfᵤ. Thus, by measuring y, we can calculate the fraction of unfolded protein: fᵤ=(y_{f}−y)/(y_{f}−yᵤ). The equilibrium constant for the unfolding process is Kᵤ=fᵤ/(1−fᵤ) and melting temperatures (Tₘ) are obtained at Kᵤ=1.

8-Anilino-1-naphtalene sulfonic acid (ANSA) Fluorescence Enhancement

Protein concentration was adjusted to 0.1 mg/ml in borate-phosphate buffer (55 mM Na₂B₄O₇, 55 mM NaH₂PO₄, pH 6.8) and equilibrated 1 hour at room temperature. ANSA was added to a final concentration of 10 μM and equilibrated 5 minutes prior to measurements. Spectra were recorded using an LS50-B Perkin-Elmer fluorometer with an excitation wavelength of 380 nm. Spectra were collected from 410 to 550 nm. Correction for buffer signal and ANSA was applied. Enhancement factors were calculated with the ratio of fluorescence intensity at 480 nm of free versus bounded ANSA.

Intrinsic Fluorescence Measurements

Protein concentration was adjusted to 0.1 mg/ml in borate-phosphate buffer and equilibrated 1 hour at room temperature. Chemical denaturation was performed by adding urea to a final concentration of 8 M and equilibrated at room temperature 5 minutes before measurements. Spectra were recorded using an LS50-B Perkin-Elmer fluorometer with an excitation wavelength of 280 nm. Spectra were collected from 295 to 360 nm and corrected for buffer emission.

Measurements were done as described in Example 1. Cytochrome C (ICN Biomedicals #101467) was used as an internal reference in order to allow for comparison of different digestion experiments.

Results and Discussion

Design Strategy

In the canonical alpha helix coiled coils folds, heptad positions b, c and f are solvent exposed. Hydrophobic side-chains at such exposed positions in native protein are known to constrain the environment to organize a hydration structure around them, which has an entropical cost that destabilizes the native form of the protein.

In MB-1, two hydrophobic side chains occupy heptad positions c, which are predicted to be exposed if folded as per design: Leu 19 and Leu 68. The substitution of these leucines by acidic residues could lead to the formation of solvent exposed salt bridges with the two nearby lysine residues at the surface of MB-1 as shown in the FIG. 17. Among the acidic residues, glutamate is the closest polar isoster of leucine in the natural occurring amino acids, which approximately limit the effect of the substitution to a change in polarity of the residue's side-chain. Glutamate has also a higher helix propensity and is a better salt-bridge former than aspartate, which is known, in some context, to behave as helix breaker.

Secondary Structure Analysis

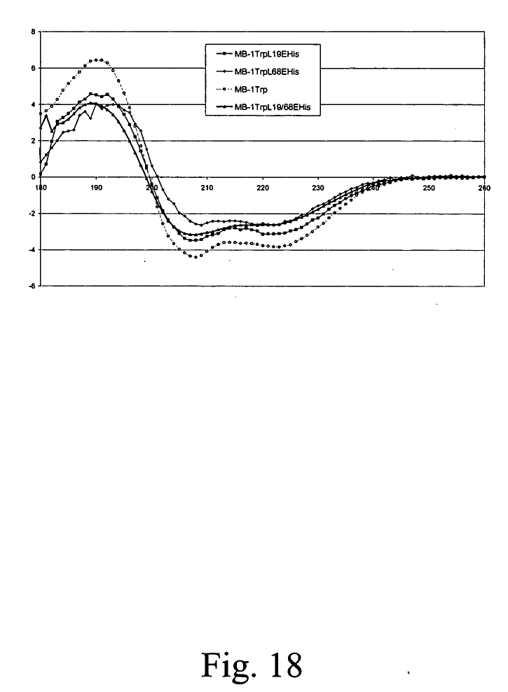

Secondary structures were monitored using far-UV circular dichroism (FIG. 18). All three mutant proteins showed the typical signature of helical proteins, similar to MB-1Trp, with a slightly lower signal. Using CDSSTR algorithm, all mutants were found to have about 40% of their amide groups to be in a helical environment (Table 2). This percentage is rather low compared to almost all previously described MB-1 mutant proteins shown to have a helical content of about 50% (Doucet et al., 2002, J. Agri. Food Chem., 50:92-98; Gagnon et al., 2000; FEBS Letters, 484:144-148). The loss in helical structure of all mutants is correlated to a comparable gain in β-structure. Note that these important changes in secondary structure are caused only by a single amino acid substitution.

TABLE 2
Calculated content of secondary structure for MB-1Trp and its variants

| Proteins | Helix ± 3% | β-Strand ± 3% | β-Turn ± 3% | Others ± 3% | Tm (° C.) ± 1° C. | Tm (° C.) with salt ± 1° C. |
|---|---|---|---|---|---|---|
| MB-1 Trp | 51 | 8 | 14 | 28 | 55.0 | — |
| MB-1 TrpL19EHis | 37 | 15 | 13 | 35 | 47.6 | 47.1 |
| MB-1 TrpL68EHis | 37 | 18 | 12 | 33 | 61.1 | 46.6 |
| MB-1 TrpL19-68EHis | 38 | 14 | 12 | 36 | 42.5 | — |

Tertiary Structure Analysis

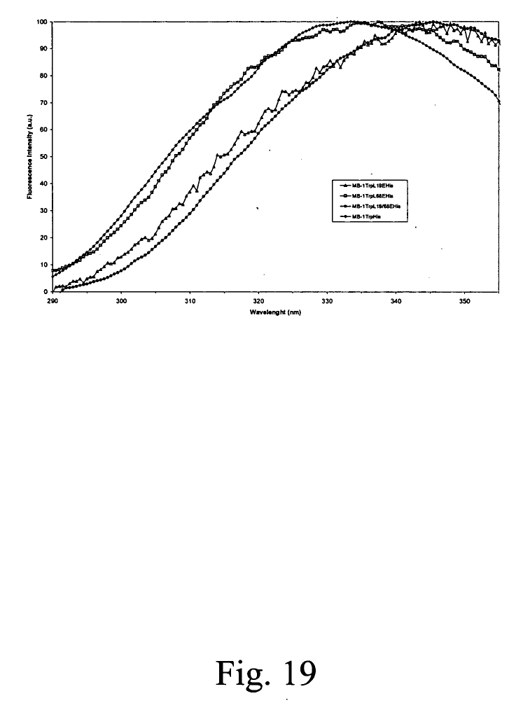

Intrinsic fluorescence measurements were performed in order to monitor the environment of the sole tryptophan residue predicted to be in the core of the protein. As seen in FIG. 19, MB-1TrpL68EHis fluorescence spectrum peaked at 335 nm with a significant shift upon chemical denaturation to 345 nm, which is reminiscent of MB-1Trp. These maxima show that Trp62 is protected in the folded state, and become solvated after denaturation. At variance, mutants L19E and L19E/L68E fluorescence spectra peaked at 345 nm with no shift upon chemical denaturation, indicating a total solvent exposure of the tryptophan side-chain in both proteins, in either native or denaturing conditions. The mutation L19E lead to an important modification of the tertiary structure, regardless of the mutation in position 68.

ANSA binding experiment were performed in order to further explore the tertiary structure of the mutant proteins. As seen in Table 3, an enhancement factor around 3 for all proteins was found (same as for the parent protein MB-1Trp) and lower than the value expected for poorly folded proteins. This indicates that even if the hydrophobic core seemed to be packed in a different way than MB-1Trp for the MB-1TrpL19EHis and MB-1TrpL19E/L68EHis mutant proteins, the three molecules are rather well folded compared to a protein in a molten-globule state.

TABLE 3
Tryptophane and ANSA fluorescence experiments of
MB-1Trp and its variants

| Proteins | λₘₐₓ Trp | Fluidity of the core |
|---|---|---|
| MB-1Trp | 333 nm | 3 |
| MB-1TrpL19EHis | 345 nm | 2.5 |
| MB-1TrpL68EHis | 335 nm | 3.3 |
| MB-1TrpL19/68EHis | 345 nm | 3 |

Effect of the Mutations on Conformational Stability

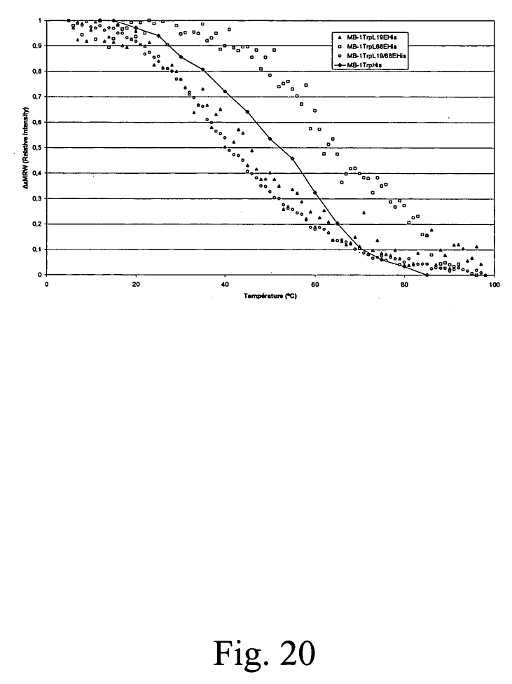

The conformational stability was assessed by monitoring the circular dichroism signal at different temperature. Denaturation curves are shown in FIG. 20 with the corresponding melting temperatures in Table 2. Mutations L19E and L19E/L68E seem to destabilize the parent molecule MB-1Trp, leading to a loss of 8.4 and 12.5° C. in melting temperature respectively. However, the mutation L68E stabilize MB-1Trp by more than 6.1° C., an improvement that makes it the most thermoresistant mutant so far in the MB-1 family. Interestingly, MB-1TrpL68EHis appears folded from 15 up to 40° C. This behavior can lead to a great improvement in proteolytic resistance in cow rumen, in which the temperature is around 39° C. Addition of 500 mM NaCl was performed in order to investigate the contribution of surface electrostatic interactions in mutants L68E and L19E. Masking charges in L68E leads to an important destabilization in agreement with the formation of a stabilizing salt bridge in this mutant. At variance, the presence of salt had no impact on MB-1L19E melting temperature, suggesting that stabilizing salt bridges between E19 and neighboring lysine were not formed.

Effect of the Mutations on Proteolytic Degradation

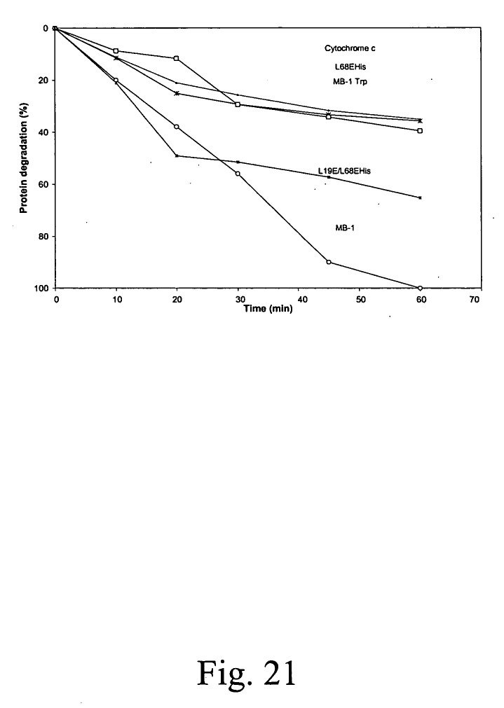

The proteolytic resistance of the three mutants was measured and compared to the parent protein MB-1Trp. As can be seen in FIG. 21, the L68E mutant resistance to degradation compared to that of MB-1Trp (the parent molecule), and to cytochrome c (a natural protein of similar size). Mutation L19E led to a destabilization of MB-1Trp, regardless of mutation in position 68. The results suggest a correlation between conformational thermostability and proteolytic resistance for all MB-1Trp proteins analysed here.

The mutation L68E was found to promote improvements in MB-1Trp characteristics, as supported by fluorescence measurements, thermal denaturation and proteolytic degradation experiments.

Contribution to stability of this mutant protein can come from two factors: 1-removal of surface hydrophobic side-chain (L68), and/or 2-formation of stabilizing salt bridge. The impact of salt on the mutant thermostability strongly suggests that a stabilizing bridge is formed indeed. Note that the stabilisation observed here is not the result of the neutralisation of the helix macrodipole, which requires insertion of negatively charged residues at the N-terminal end of helices.

The mutation L19E led to destabilization of MB-1Trp protein. Adding salt had no effect on thermal denaturation, suggesting that there were no stabilizing contributions from salt bridges. More importantly, the mutation had a drastic impact on tertiary fold, leading to tryptophan exposure and loss of thermostability. Surprisingly, while two positive modifications at the surface of the mutant L19E were expected: 1-removal of a non-polar leucine from its surface and 2-formation of stabilizing electrostatic interactions; negative results were recorded, suggesting a major, destabilizing impact on the protein fold. Interestingly, it appears that the stabilizing mutation L68E has no effect when combined with the disrupting mutation L19E. It seems that the structural destabilization provoked by L19E mutation cannot be corrected by the other mutation L68E.

### EXAMPLE V

### Control of Proteolytic Degradation of the Methionine Enriched MB-1Trp Protein

Materials and Methods

MB-1Trp Expression and Purification

MB-1Trp was expressed and purified according to the method described herein. Protein samples intended for mass spectrometry were purified on a DEAE-Sepharose column, equilibrated with 10 mM Tris-1 mM EDTA containing 0.1% Tween-20. The protein was eluted using a continuous NaCl gradient (0 to 1000 mM). Fractions were analysed by sodium dodecyl sulphate-polyacrylamide gel electrophoresis (SDS-PAGE) using 16% polyacrylamide-tricine gels and colored by silver nitrate staining (Amersham Biosciences). The proteins were concentrated and dialysed as described previously in Example I.

Matrix Assisted Laser Desorption Ionisation-Time of Flight (MALDI-TOF) Mass Spectrometry

The size of the fragments was determined using an Applied Biosystems MALDI-TOF Mass Spectrometer Voyager System 2016 with an α-cyano-4-hydroxycinnamic acid matrix. WinPep software (Hennig, L. 1999, BioTechniques 26:1170-1172) was used to calculate the mass of the fragments as deduced from sequence.

MB-1TrpHis Construction, Expression and Purification

To allow a C-terminal specific purification of MB-1Trp and its fragments, and detection with a commercially available anti-His-Tag monoclonal antibody, a poly-histidine tag (His-tag) was ligated to the C-terminal end of MB-1Trp. This was achieved by constructing a chimeric plasmid from the original pMal-c2 MB-1Trp and pMal-c2 MB-1His plasmids (Grundy et al. 1998, Protein Exp. Purif. 13:61-66). Protein samples for N-terminal sequencing were purified by amylose affinity chromatography followed by an immobilized metal affinity chromatography (IMAC), as described earlier (Grundy et al. 1998, Protein Exp. Purif. 13:61-66).

N-Terminal Sequencing

Proteins were transferred to a polyvinylidene fluoride (PVDF) membrane as described by Geisow and Aitken (Geisow et al. 1989, In: Findlay, J. B. C. and Geisow, M. J. (Eds), IRL Press, Oxford, pp. 85-98). The proteins were then sequenced using an ABI model 473A Protein Sequencer (Service Protéomique de l'Est du Québec, Sainte-Foy, Canada).

Preparation of MB-1TrpHis Mutants

Substitutions of asparagine in position 44 and leucine in position 45 of MB-1TrpHis were performed using the “Site-Directed Mutagenesis Kit” (Stratagene). The asparagine codon (AAC) was replaced by a GA(A/T) codon, encoding for E or D. The leucine codon (CTG) was replaced by a (A/T/G)T(G/C) codon, encoding V, M, I, L and F in the following proportion: 2:1:1:1:1 respectively. A total of ten different mutation sets were therefore available with this strategy. The mutagenic oligonucleotides, purchased from Biocorp inc. (Montreal, Canada), are shown below along with the corresponding MB-1TrpHis original sequence. Oligonucleotides were purified using polyacrylamide gel electrophoresis and phosphorylated.

| | | |
|---|---|---|
| MB-1TrpHis | 5′-CAATG AAG AAT CAT CTT | (SEQ ID NO:28) |
| | CAA AAC CTG ATG CAG AAG | |
| | ACT AAG AAC-3′ | |
| Oligo 9: | 5′-CAATG AAG AAT CAT CTT | (SEQ ID NO: 29) |
| | CAA GAW DTS ATG CAG AAG | |
| | ACT AAG AAC-3′ | |
| Oligo 10: | 5′-GTT CTT AGT CTT CTG | (SEQ ID NO: 30) |
| | CAT SAH WTC TTG AAG ATG | |
| | ATT CTT CATTG-3′ | |

where W stand for A or T; D stand for A, T or G; S stand for C or G; and H stand for A, C, or T.

The mutations were confirmed by dideoxynucleotide DNA sequencing. The mutated genes were expressed in fusion with the Maltose-Binding Protein (MBP) in pMal-c2 expression vectors (New England Biolabs).

Proteolytic Resistance Screening by Western Blotting

E.coli XL-1Blue cells (recA1, endA1, gyrA96, thi-1, hsdR17(r_{K}^{-,m}_{K}⁺), supE44, relA1, lac⁻, [F′ proAB, lacI^{q}ZΔ M15, Tn10(Tet^{r})]) (Stratagene, Cedar Creek, USA) harbouring pMal-c2 MB-1TrpHis mutant plasmids were grown in 15 mL of LB Miller medium (EM Science, Hawthorne, USA) supplemented by 100 mg 1-1 ampicillin (Sigma) to an optical density of 0.3 at 600 nm. Transcription was induced by adding 1 mM isopropylthio-β-D-galactoside (IPTG) and cells were grown for an additional 3 hours. Cells were then centrifuged at 4000 g for 10 minutes and resuspended in 5 ml cleavage buffer (20 mM Tris, 100 mM NaCl and 3 mM CaCl₂). A 30 seconds sonication (60% output control on a Branson Sonifier 250) was performed to break the cell wall. To 10 μl aliquots of broken cells, 3 μl of factor Xa (New England Biolabs) and 10 μl of cleavage buffer were added, followed by an overnight incubation at 4° C. in order to cleave MB-1 variants from their MBP fusion. The solutions were then incubated at 37° C. in order to reactivate E.coli proteases and challenge the mutants' resistance to proteolytic degradation. Following 0 and 30 minutes of incubation with E.coli proteases, the solutions were denatured using 2% SDS and heated at 95° C. for 5 minutes. Samples were analysed by a SDS-PAGE followed by an overnight transfer onto a nitrocellulose membrane (#162-0146, Bio-Rad, Hercules, USA) following the manufacturer's instructions. The Western blotting was performed using a mouse anti-His-Tag monoclonal antibody (Novagen, Darmstadt, Germany) and a goat anti-mouse secondary antibody conjugated to alkaline phosphatase. The revelation of the binding was performed by a standard colorimetric Western blotting procedure (Novagen).

Expression and Purification of MB-1TrpHis Mutants

All mutants were expressed and purified as described above, with a second chromatography involving metal affinity purification using the modifications cited above.

Protein Quantification and Purity Evaluation

For all experiments, protein concentration was determined by bicinchoninic acid (BCA) assay (Sigma) using bovine serum albumin as a standard. SDS-PAGE experiments were conducted prior to measurements to confirm protein purity.

Conformational Investigation by Circular Dichroism (CD)

CD measurements were carried out as described in Example I. Experiments were conducted at a protein concentration of 0.6 mg ml-1 in a phosphate buffer (128 mM NaH₂PO₄, pH6.8).

Thermal Denaturation

The samples were prepared as described in the previous section and conformational changes were monitored by CD. To measure thermostability, temperature was increased from 5 to 98° C. at a rate of 20° C. h-1 using a Neslab™ RTE-111 controlled by Jasco™ spectropolarimeter software. CD spectra were collected at every 5° C. from 190 to 260 nm, at a speed of 20 nm min-1, and CD signals at 222 nm were collected at every 1° C. Thermal stability was calculated assuming a unimolecular, two-state process as previously described in Pace et al.(1989, In: Creighton, T. E. (ed.), IRL. Press, Oxford, pp. 311-330).

The CD signal at 222 nm measured at various temperatures was used as the property (y) indicative of the extent of unfolding. In the folded state, the parameter y=y_{f} and the fraction of folded protein f_{f} is equal to 1. When the protein is unfolded, the parameter y=Yᵤ, and the fraction of unfolded protein fᵤ is equal to 1. For intermediate states, y is given by y_{f}f_{f}+yᵤfᵤ. Thus, by measuring y, we can calculate the fraction of unfolded protein: fᵤ=(y_{f}−y)/(y_{f}−yᵤ). The equilibrium constant for the unfolding process is Kᵤ=fᵤ/(1−fᵤ) and melting temperatures (Tₘ) are obtained at Kᵤ=1 (Pace et al. 1989, In: Creighton, T. E. (ed.), IRL. Press, Oxford, pp. 311-330).

Results and Discussion

Identification of the Protease Sensitive Peptide Bonds

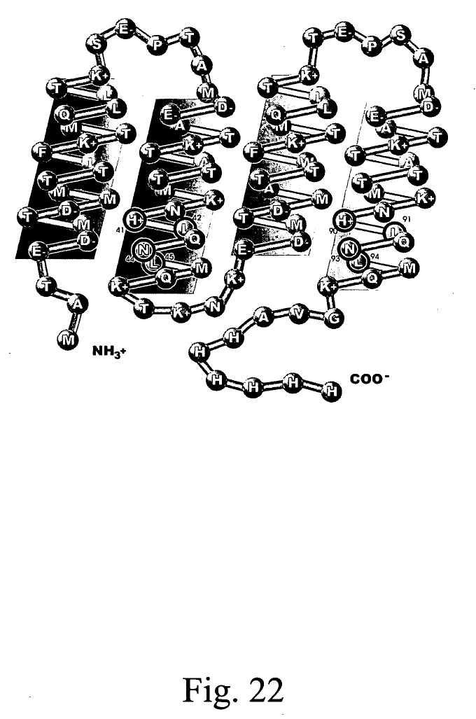

Mass spectrometry was performed on MB-1Trp fragments purified using anionic exchange purification. The assignment of mass spectrometry results is described in Table 4. Seven fragments were tentatively assigned, with the most important being M1-N93 (peak no. 1) and M1-N44 (peak no. 3). For some fragments slight differences in calculated and measured masses were found. They might be ascribed to post-translational modifications, such as methionine oxidation, which is not unexpected considering the high methionine contents of MB-1Trp. The assignment of peaks no. 1 and 3 was confirmed by N-terminal sequencing. To facilitate the fragment identification process, N-terminal sequencing was performed on MB-1 TrpHis fragments recovered from an IMAC column. Only fragments that carry the intact C-terminus are retained under such conditions. After recovery from E. coli, several fragments were obtained, the two most important bearing the sequence: LMQKTKNKE and LMQKGVAH at their N-terminus respectively. Such sequences corresponds to amino acids 45-53 and 94-101 respectively. Therefore, both approaches (mass spectrometry and N-terminal sequencing) point to a cleavage between N44 and L45, and a second site at N93 and L94. The detection of the second site is not unexpected, because in MB-1Trp, helices 2 and 4 are predicted equivalent regarding both conformation and sequence (per design, see FIG. 22). Furthermore, the correlation between fragments size and N-terminal sequencing results suggest that adding the His tag to MB-1Trp did not change its sensitivity to proteases.

TABLE 4
Mass spectrometry analysis of MB-1Trp proteolytic fragments

| | Mass | Relative Intensity | | Calculated Mass |
|---|---|---|---|---|
| Peak no. | (Da) | (a.u.) | Fragment | (Da) |
| 1 | 10674 | 65 | M1-N93 | 10663 |
| 2 | 10318 | 30 | M1-H90 | 10308 |
| 3 | 5002 | 75 | M1-N44 | 5000 |
| 4 | 4645 | 25 | M1-H41 | 4644 |
| 5 | 6775 | 15 | L42-A100 | 6765 |
| 6 | 5331 | 20 | L45-H90 | 5326 |

Another minor fragment had the amino-terminus sequence LQNLMQ, which corresponds to either segment 42-47 associated to peak no. 5 (L42-A 100) or 91-96, associated to peak no. 2 and 6 (M1-H90 and L45-H90 respectively). The population of fragment that has been cut using the site N44-L45 (Fragment M1-N44, peak no.3) was two-fold more important than the ones being cut at the site H41-L42 (Fragment M1-H41 peak no. 4 and fragment L42-A100, peak no. 5). Similarly, the fragment population of M1-N93 (peak no. 1) was three-fold more important than the M1-H90 population (peak no. 2). Thus, the N-L bonds appear to be much more susceptible or accessible than H-L bonds, probably because they are more exposed as predicted per design (FIG. 22). Among the two N-L bonds identified, the N44-L45 bond was selected for our study. A cleavage right in the middle of the protein was believed to have a more important impact on protein integrity and “residual” resistance after a first cleavage.

Combinatorial Mutagenesis

The predicted positions of the two amino acids targeted by proteolysis (N44, L45) are shown in FIG. 22. It was hypothesised that changing the residues directly involved at this peptide bond would decrease sensibility to a putative specific protease of E.coli. The N44 residue was allowed to be mutated to D or E, two acidic residues. Interactions with the neighbouring basic residues, H41 and K48, could promote the formation of stabilizing intra-helical salt-bridges. The structural stabilisation of this region of helix 2 could also contribute to prevent proteolytic activity.

The L45 residue was allowed to be mutated to the hydrophobic amino acids, I, V, M, F and the wild-type L. With this strategy, we hoped that the most accommodating residues would result in more stable mutants while providing a change in the amino acid sequence at the target site. Since L45 is predicted to be buried in the hydrophobic core, where mutations are usually not well tolerated, a wide variety of non-polar amino acids was allowed.

Clones generated by the mutagenesis reaction were sequenced to assess the distribution of the mutations in our bank. The results are shown in Table 5. Seven out of 10 possible combinations were recovered from the mutant library. Interestingly, both possible mutant with a conserved L in position 45 were absent from the clones sampled for sequencing. There was no obvious bias in the distribution of the mutations.

TABLE 5
Mutations distribution in the MB-1TrpHis mutants library

| Mutations in Position 44-45 | Number of Clones Sequenced |
|---|---|
| E-V | 1 |
| E-M | 2 |
| E-F | 1 |
| D-V | 3 |
| D-M | 2 |
| D-F | 3 |
| D-I | 2 |

Screening Mutants for Proteolytic Resistance

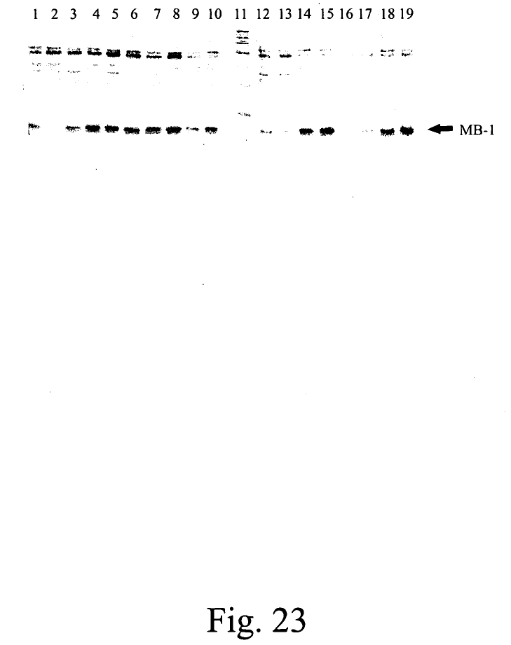

Randomly selected mutants were screened for their proteolytic resistance by exposing them to proteolytic degradation assays, followed by an analysis by Western blotting with an anti-His-Tag monoclonal antibody. For degradation assays, MB-1TrpHis (containing a His tag) was used as the “wild type” protein instead of MB-1Trp. As can be seen on FIG. 23, several mutants outperformed their parental counterpart MB-1 TrpHis when exposed to E.coli proteases: The disappearance of MB-1TrpHis after a 30 minutes incubation at 37° C. is evidenced by the comparison of the band intensities in lanes 1 vs 2, and 12 versus 13. Most of the 7 mutants analysed outperformed the protein MB-1TrpHis in terms of relative amount of protein left after incubation.

The screened clones were sequenced to assess the distribution of the stabilizing mutations. The most stabilized mutants carry the mutation combinations E-V, E-M, D-I, D-V and D-M. The least stable mutants carry the mutation combinations E-F (lanes 16-17) and D-F (lanes 9-10). These results could be explained by the fact that the hydrophobic core might not be able to harbour the large phenylalanine side-chain in position 45, which could promote local unfolding of the protein and expose protease targets.

Careful examination of FIG. 23 allows to detect several protein bands that differ from MB-1TrpHis and its mutants (band position shown by an arrow). In the upper part of the gels, the first band at the top is the MBP-MB-1TrpHis fusion protein that escaped cleavage with factor Xa, migrating at 54 kDa. The next band is the large cleavage product of the fusion: MBP, running at 43 kDa. Then, few bands in the mid-range (25-35 kDa) are believed to be E. coli proteins with histidines at their surface, resulting in some affinity for the antibody used in this experiment.

Smaller protein fragments (probably MB-1TrpHis proteolytic products) were detected in the parent protein electrophoretic pattern and for most mutants. They appear to be less important for the mutants however, when considering the relative amount of full length product versus degradation bands.

Secondary structures of the two most stable mutants MB-1TrpHis N44E/L45V and MB-1TrpHis N44E/L45M were analysed by CD. The spectra measured for the mutants were typical of helical proteins and equivalent to MB-1Trp. The two mutants' secondary structures were calculated using CDSSTR algorithm as shown in Table 6. No significant changes in the secondary structures were detected, which indicates that proteolytic resistance is not the consequence of conformational changes that would promote hindrance for protease access to its target.

Effect of Mutations on Conformational Stability

Thermal denaturation experiments were carried out on the two purified mutants using CD as described above in Materials and Methods. Their melting temperatures (Tm) are indicated in Table 6. Each mutant was as thermostable as MB-1 TrpHis indicating that the mutations led to no major increase in structural stability.

TABLE 6
Conformational analysis of MB-1TrpHis and its mutants

| Proteins | Helix ± 3% | β-Strand ± 3% | β-Turn ± 3% | Others ± 3% | Tm (° C.) ± 1° C. |
|---|---|---|---|---|---|
| MB-1TrpHis | 45 | 12 | 12 | 31 | 55.0 |
| E44-M45 | 46 | 10 | 12 | 32 | 52.0 |
| E44-V45 | 48 | 12 | 11 | 29 | 53.5 |

We reported in the present example a continuation of our approach to designing stable proteins with the additional constraint represented by a biased content in selected EAA (M, T, K and L). The mutations introduced to the protein MB-1Trp were shown to have an important impact on its resistance to E.coli proteases. Two mutant proteins were still detectable by Western blotting after two hours of incubation with E.coli proteases, whereas under similar experimental conditions, no MB-1TrpHis was detectable after 30 minutes of incubation. This alone is a major improvement in MB-1Trp behaviour. In order to understand the nature of the stabilization brought by the mutations, their effect on MB-1Trp conformation was assessed. As shown by CD and thermal denaturation experiments, these contributions appeared marginal. This implies that the improvement most probably came from the simple deletion of the targeted sequence recognised by the protease(s).

As the targeted bonds were identified to be the same in second and fourth helices, it gives us evidence that the structure around the homologous site Asn93-Leu94 could be quite similar and that our strategy, applied to these two positions, would similarly improve the behavior of MB-1Trp, at least for the yield of full length protein.

The present strategy of protease target location reveals itself to be an effective and simple way to increase MB-1Trp half-life in a normal cellular environment.

### EXAMPLE VI

### Sustitution of Aniline Residues in Hydrophobe Core

Materials and Methods

Construction of the Library and Protease-Resistant Proteins

In order to be able to clone the MB-1TrpHis library in the T7Select10-3 phage display system (Novagen), an EcoR1 restriction site had to be engineered in the plasmid pMAL-c2MB-1TrpHis using Quickchange Kit (Stratagene). This site allowed the fusion of our mutant proteins with p10 T7 major coat protein.

The library was prepared using this engineered plasmid. MB-1TrpHis variants were prepared by randomizing five alanine positions (31, 35, 59, 80 and 84) located in the hydrophobic core around a tryptophan residue. Each position was allowed to be substituted by F, I, L, M and V in the proportion 1:1:1:1:2. The mutagenesis was performed using 75 cycles of sexual PCR with 2 sets of 2 oligonucleotides that degenerate the five positions:

Terminal primers were added to the sexual PCR and an extra 30 cycles of PCR were performed to amplified the full length product. The mutagenic reaction was sequenced using dideoxynucleotide method in order to confirm the presence of the degeneracy in the predicted positions. The 400 bp was gel purified, digested using EcoRI and HindIII and cloned in the T7Select10-3 phage display system.

Panning of the Library

After a first round of amplification made following the producer's conditions, the library undergoes five round of proteolysis-based selection. Selection was made following the protocol described by Funicane and Woolfson (Finucane and Woolfson, 1999, Biochemistry 38:11613-11623) with these minor modifications. Proteases treatments were preceded by an heat treatment of 65° C. for 15 minutes. Pronase E (Sigma) was used at 0.675 mg/ml for 45 minutes at 39° C. instead of chymotrypsin. Recovered phages were amplified using the same method cited earlier.

Overexpression of Proteins

Selected phages carrying a variant of MB-1TrpHis were amplified using PCR and cloned back into pMAL-c2 vector. The resulting plasmids were transformed into super-competent E.coli X1-1Blue cells. Protein expression was conducted as described above.

It is to be noted that all document cited herein has been incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for improving supplying of at least one essential amino acid to a human or an animal or endowing a human or an animal with at least one essential amino acid, comprising administering to said human or animal at least one polypeptide or a nucleic acid sequence encoding said polypeptide, said polypeptide having amino acid sequence consisting of at least one of SEQ ID NO:1 to SEQ ID NO:5 or a fragment thereof, in which at least one leucine at position 19, 45 or 68, one asparagine at position 44, or one alanine at position 31, 35, 59, 80, or 84 of said at least one SEQ ID NO:1 to SEQ ID NO:5 is substituted with a different amino acid.

2. The method of claim 1, wherein said at least one leucine is substituted with a glutamic acid, an aspartic acid, a valine, an isoleucine, a methionine, or a phenylalanine, said asparagine is substituted with a glutamic acid, or said alanine is substituted with a valine, an isoleucine, a phenylalanine, a leucine, or a methionine.

3. The method of claim 1, wherein said functional fragment comprises between 10 and 90 amino acids.

4. The method of claim 1, wherein said administration is an oral administration.

5. The method of claim 1, wherein said nucleic acid sequence is a DNA or a RNA sequence.

6. The method of claim 1, wherein said SEQ ID NO:1 to SEQ ID NO:5 have additionally at least one tryptophane at position 62, leucine at position 13 or 91, or methionine at position 10 or 87 substituted with another amino acid.

7. The method of claim 6, wherein said tryptophane is substituted with a tyrosine, said leucine at position 13 or 91 is substituted with a cysteine and said methionine is substituted with a cysteine.

8. A composition for improving supplying of at least one essential amino acid to a human or an animal or endowing a human or an animal with at least one essential amino acid, said composition comprising at least one polypeptide having amino acid sequence consisting of at least one of SEQ ID NO:1 to SEQ ID NO:5 or a fragment thereof, or a nucleic acid sequence encoding said polypeptide or fragment thereof, having at least one substituted amino acid as described in claim 1.

9. The composition of claim 8, wherein said essential amino acid is a methionine, lysine, threonine, leucine, tryptophan, arginine, an analog or a derivative thereof.

10. The composition of claim 8, wherein said SEQ ID NO:1 to SEQ ID NO:5 have additionally at least one tryptophane at position 62, leucine at position 13 or 91, or methionine at position 10 or 87 substituted with another amino acid.

11. The composition of claim 10, wherein said tryptophane is substituted with a tyrosine, said leucine at position 13 or 91 is substituted with a cysteine and said methionine is substituted with a cysteine.

12. A polypeptide consisting of an amino acid sequence selected from the group consisting of SEQ ID NO:1 to SEQ ID NO:5 or a functional fragment thereof, in which at least one leucine at position 19, 45 or 68, one asparagine at position 44, or one alanine at position 31, 35, 59, 80, or 84 of at least one of said SEQ ID NO:1 to SEQ ID NO:5 is substituted with a different amino acid.

13. The polypeptide of claim 12, wherein said essential amino acid is a methionine, lysine, threonine, leucine, tryptophan, arginine, an analog or a derivative thereof.

14. The polypeptide of claim 12, wherein said SEQ ID NO:1 to SEQ ID NO:5 have additionally at least one tryptophane at position 62, leucine at position 13 or 91, or methionine at position 10 or 87 substituted with another amino acid.

15. The polypeptide of claim 14, wherein said tryptophane is substituted with a tyrosine, said leucine at position 13 or 91 is substituted with a cysteine and said methionine is substituted with a cysteine.

16. A nucleotide sequence encoding for a polypeptide as described in claim 13.

17. The nucleotide sequence of claim 16 being a DNA or a RNA sequence.

18. A nucleotide sequence selected from the group consisting of, SEQ ID NO:6 to SEQ ID NO:10, in which at least one codon is mutated to allow synthesis from said nucleotide sequence a polypeptide as described in claim 12.

19. An expression vector comprising a nucleotide sequence as described in claim 12.

20. An expression vector comprising a nucleotide sequence as described in claim 18.

21. A cell transformed with the expression vector as described in claim 19.

22. A cell transformed with the expression vector as described in claim 20.

23. A method for improving physical, biochemical or biological property of a polypeptide having amino acid sequence consisting of at least one of SEQ ID NO:1 to SEQ ID NO:5 or a fragment thereof, comprising substituting at least one leucine at position 19, 45 or 68, one asparagine at position 44, or one alanine at position 31, 35, 59, 80, or 84 of at least one of said SEQ ID NO:1 to SEQ ID NO:5 with a different amino acid.

24. The method of claim 23, wherein said at least one leucine is substituted with a glutamic acid, an aspartic acid, a valine, an isoleucine, a methionine, or a phenylalanine, said asparagine is substituted with a glutamic acid, or said alanine is substituted with a valine, an isoleucine, a phenylalanine, a leucine, or a methionine.

25. The method of claim 23, wherein said property is resistance to temperature or to proteolysis.

26. The polypeptide of claim 23, wherein said essential amino acid is a methionine, lysine, threonine, leucine, tryptophan, arginine, an analog or a derivative thereof.

27. The polypeptide of claim 23, wherein said SEQ ID NO:1 to SEQ ID NO:5 have additionally at least one tryptophane at position 62, leucine at position 13 or 91, or methionine at position 10 or 87 substituted with another amino acid.

28. The polypeptide of claim 27, wherein said tryptophane is substituted with a tyrosine, said leucine at position 13 or 91 is substituted with a cysteine and said methionine is substituted with a cysteine.


SEQUENCE LISTING (30 sequences)

SEQ ID NO: 1 - protein, 100 aa, Recombinant molecule
  VARIANT (1)...(100): MB-1Trp
MATEDMTDMMTTLFKTMQLLTKSEPTAMDEATKTATTMKNHLQNLMQKTKNKEDMTDMATTWFKTMQLLTKTEPSAMDEATKTATTMKNHLQNLMQKGVA

SEQ ID NO: 2 - protein, 100 aa, Recombinant molecule
  VARIANT (1)...(0): MB-1TrpRH
MATEDMTDMCTTLFKTMQLLTKSEPTAMDEATKTATTMKNHLQNLMQKTKNKEDMTDMATTWFKTMQLLTKTEPSAMDEATKTATTMKNHCQNLMQKGVA

SEQ ID NO: 3 - protein, 100 aa, Recombinant molecule
  VARIANT (1)...(100): MB-1LH
MATEDMTDMMTTCFKTMQLLTKSEPTAMDEATKTATTMKNHLQNLMQKTKNKEDMTDMATTYFKTMQLLTKTEPSAMDEATKTATTCKNHLQNLMQKGVA

SEQ ID NO: 4 - protein, 100 aa, Recombinant molecule
  VARIANT (1)...(100): MB-1RH
MATEDMTDMCTTLFKTMQLLTKSEPTAMDEATKTATTMKNHLQNLMQKTKNKEDMTDMATTYFKTMQLLTKTEPSAMDEATKTATTMKNHCQNLMQKGVA

SEQ ID NO: 5 - protein, 100 aa, Recombinant molecule
  VARIANT (1)...(100): MB-1TrpLH
MATEDMTDMMTTCFKTMQLLTKSEPTAMDEATKTATTMKNHLQNLMQKTKNKEDMTDMATTWFKTMQLLTKTEPSAMDEATKTATTCKNHLQNLMQKGVA

SEQ ID NO: 6 - DNA, 303 bp, Recombinant molecule
  gene (1)...(303): MB-1Trp
atggctacggaagacatgaccgacatgatgaccaccctgtttaaaactatgcagctgttgaccaagtcggaacccacggctatggacgaggccactaaaacggctactacaatgaagaatcatcttcaaaacctgatgcagaagactaagaacaaagaagacatgacggacatggccactacgtggttcaaaacgatgcagttgttaacgaagaccgacccctcggccatggacgaggccacgaagacggctacaaccatgaaaaatcatctgcagaacttgatgcaaaaaggcgtagcttaa

SEQ ID NO: 7 - DNA, 302 bp, Recombinant molecule
  gene (1)...(302): MB-1TrpRH
atggctacggaagacatgaccgacatgtgcaccaccctgtttaaaactatgcagctgttgaccaagtcggaacccacggctatggacgaggccactaaaacggctactacaatgaagaatcatcttcaaaacctgatgcagaagactaagaacaaagaagacatgacggacatggccactacgtgcttcaaaacgatgcagttgttaacgaagaccgagccctcggccatggacgaggccacgaagacggctacaaccatgaaaaatcattgccagaacttgatgcaaaaaggcgtagctta

SEQ ID NO: 8 - DNA, 303 bp, Recombinant molecule
  gene (1)...(3): MB-1LH
atggctacggaagacatgaccgacatgatgaccacctgctttaaaactatgcagctgttgaccaagtcggaacccacggctatggacgaggccactaaaacggctactacaatgaagaatcatcttcaaaacctgatgcagaagactaagaacaaagaagacatgacggacatggccactacgtacttcaaaacgatgcagttgttaacgaagaccgagccctcggccatggacgaggccacgaagacggctacaacctgcaaaaatcatctgcagaacttgatgcaaaaaggcgtagcttaa

SEQ ID NO: 9 - DNA, 303 bp, Recombinant molecule
  gene (1)...(303): MB-1RH
atggctacggaagacatgaccgacatgtgcaccaccctgtttaaaactatgcagctgttgaccaagtcggaacccacggctatggacgaggccactaaaacggctactacaatgaagaatcatcttcaaaacctgatgcagaagactaagaacaaagaagacatgacggacatggccactacgtacttcaaaacgatgcagttgttaacgaagaccgagccctcggccatggacgaggccacgaagacggctacaaccatgaaaaatcattgccagaacttgatgcaaaaaggcgtagcttaa

SEQ ID NO: 10 - DNA, 303 bp, Recombinant molecule
  gene (1)...(303): MB-1TrpLH
atggctacggaagacatgaccgacatgatgaccacctgctttaaaactatgcagctgttgaccaagtcggaacccacggctatggacgaggccactaaaacggctactacaatgaagaatcatcttcaaaacctgatgcagaagactaagaacaaagaagacatgacggacatggccactacgtggttcaaaacgatgcagttgttaacgaagaccgagccctcggccatggacgaggccacgaagacggctacaacctgcaaaaatcatctgcagaacttgatgcaaaaaggcgtagcttaa

SEQ ID NO: 11 - DNA, 27 bp, Artificial Sequence
  MB-1-11
atggccactacgtacttcaaaacgatg

SEQ ID NO: 12 - DNA, 27 bp, Artificial Sequence
  Tyr62Trp
atggccactacgtggttcaaaacgatg

SEQ ID NO: 13 - DNA, 27 bp, Artificial Sequence
  MG-1-13
atgatgaccaccctgtttaaaactatg

SEQ ID NO: 14 - DNA, 27 bp, Artificial Sequence
  Oligo 1 L13C
atgatgaccacctgctttaaaactatg

SEQ ID NO: 15 - DNA, 27 bp, Artificial Sequence
  MB-1-15
acggctacaaccatgaaaaatcatctg

SEQ ID NO: 16 - DNA, 27 bp, Artificial Sequence
  Oligo 2 M87C
acggctacaacctgcaaaaatcatctg

SEQ ID NO: 17 - DNA, 27 bp, Artificial Sequence
  MB-1-17
atgaccgacatgatgaccaccctgttt

SEQ ID NO: 18 - DNA, 27 bp, Artificial Sequence
  Oligo 3 M10C
atgaccgacatgtgtaccaccctgttt

SEQ ID NO: 19 - DNA, 26 bp, Artificial Sequence
  MB-1-19
atgaaaaatcatctgcagaacttgat

SEQ ID NO: 20 - DNA, 27 bp, Artificial Sequence
  Oligo 4 L91C
atgaaaaatcattgccagaacttgatg

SEQ ID NO: 21 - DNA, 24 bp, Artificial Sequence
  MB-1-21
atggccactacgtacttcaaaacg

SEQ ID NO: 22 - DNA, 24 bp, Artificial Sequence
  Oligo 5 Y62W
atggccactacgtggttcaaaacg

SEQ ID NO: 23 - DNA, 30 bp, Artificial Sequence
  MB-1 Trp
tttaaaactatgcagctgttgaccaagtcg

SEQ ID NO: 24 - DNA, 30 bp, Artificial Sequence
  Oligo 6 L19E
tttaaaactatgcaggaattgaccaagtcg

SEQ ID NO: 25 - DNA, 43 bp, Artificial Sequence
  MB-1Trp25
cgtggttcaaaacgatgcagttgttaacgaagaccgagccctc

SEQ ID NO: 26 - DNA, 43 bp, Artificial Sequence
  Oligo 7 L68E
cgtggttcaaaacgatgcaggagttaacgaagaccgagccctc

SEQ ID NO: 27 - DNA, 43 bp, Artificial Sequence
  Oligo 8 L68E
gagggctcggtcttcgttaactcctgcatcgttttgaaccacg

SEQ ID NO: 28 - DNA, 44 bp, Artificial Sequence
  MB-1 TrpHis-28
caatgaagaatcatcttcaaaacctgatgcagaagactaagaac

SEQ ID NO: 29 - DNA, 44 bp, Artificial Sequence
  Oligo 9
caatgaagaatcatcttcaagawdtsatgcagaagactaagaac

SEQ ID NO: 30 - DNA, 44 bp, Artificial Sequence
  Oligo 10
gttcttagtcttctgcatsahwtcttgaagatgattcttcattg